(19) United States
(12) Patent Application Publication
(10) Pub. No.: US 2026/0202747 A1
(43) Pub. Date: Jul. 16, 2026

(54) PHOTOSENSITIVE COMPOSITION FOR ORGANIC EL DISPLAY DEVICE, AND ORGANIC EL DISPLAY DEVICE

(71) Applicants: artience Co., Ltd., Tokyo (JP); TOYO VISUAL SOLUTIONS CO., LTD., Tokyo (JP); TOPPAN HOLDINGS INC., Tokyo (JP)

(72) Inventors: Taketo KOGURE, Chuo-ku, Tokyo (JP); Mikako HIGASHI, Taito-ku, Tokyo (JP); Yuki HIROTA, Taito-ku, Tokyo (JP)

(73) Assignees: artience Co., Ltd., Tokyo (JP); TOYO VISUAL SOLUTIONS CO., LTD., Tokyo (JP); TOPPAN HOLDINGS INC., Tokyo (JP)

(21) Appl. No.: 18/873,734

(22) PCT Filed: Jun. 21, 2023
(86) PCT No.: PCT/JP2023/022908

§ 371 Date: Dec. 11, 2024

(30) Foreign Application Priority Data
Jun. 23, 2022 (JP) 2022-101262

(51) Int. Cl. G03F 7/031; C07D 209/86

(52) U.S. Cl. CPC G03F 7/031; C07D 209/86

(57) ABSTRACT

Provided are: a photosensitive composition for organic EL display device, the photosensitive composition being excellent in preservation stability as a photosensitive composition, being excellent in curability at a low temperature, having good developability, and being capable of forming a cured film having high light-shielding properties; and an organic EL display device comprising a cured film having high light-shielding properties. This photosensitive composition for organic EL display device comprises carbon black, a polymerizable compound, a photopolymerization initiator, and a photosensitive resin, wherein the photopolymerization initiator includes a compound having two or more oxime esters in one molecule; the polymerizable compound includes a hexafunctional polymerizable compound and a trifunctional polymerizable compound; and the content of the carbon black is 30% by mass to 50% by mass in the non-volatile content of the photosensitive composition for organic EL display device.

### TECHNICAL FIELD

The present invention relates to a photosensitive composition for organic EL display device, and an organic EL display device.

### DESCRIPTION OF RELATED ART

In recent years, the development and mass production of display devices using organic EL (organic electroluminescence), which is a self-luminous element, have been progressing in electronic devices such as mobile phones and portable information terminals to meet the demands for thin, lightweight, and curved displays. Among these, applications to high-definition and small-sized displays of about 0.5 inches (hereinafter referred to as micro displays) such as head-mounted displays and electronic viewfinders are expected to take advantage of the thin and lightweight characteristics.

There are two main display systems for organic EL displays: one system involves forming a white light-emitting layer and using color filters to express colors such as RGB, and the other system involves depositing light-emitting layers that emit respective colors such as RGB to express colors. For micro displays, due to the fine pixels, it is very difficult to use the system that selectively forms RGB light-emitting layers. Therefore, the system using color filters with a white light-emitting layer is actively adopted for micro displays.

Since the pixel size of the above-mentioned micro displays is as fine as 1 μm to 5 μm, the conventionally adopted system of creating organic EL elements and color filters on separate substrates and bonding them together may result in reduced accuracy and color misalignment. Therefore, a system of forming color filters on organic EL elements has been proposed. In addition, micro displays typically have a patterned light-shielding film, such as a black matrix, formed to prevent color mixing of light from organic EL elements that passes through color filter pixels. Furthermore, a light-shielding portion may be formed in the outer peripheral portion of the display area of the element to suppress reflected light from the wiring. One of the methods for forming such light-shielding layer patterns, such as black matrix and light-shielding portion, is the photolithography method using a photosensitive composition.

For example, Patent Document 1 discloses a photosensitive resin composition which includes a binder resin, a photopolymerizable compound, a photopolymerization initiator, a colorant including a black pigment and other organic pigments, and a thermosetting compound, as a photosensitive composition for black matrix (Patent Document 1).

### RELATED ART DOCUMENT

### Patent Document

Patent Document 1: Japanese Patent Application Laid-Open No. 2019-61216

### SUMMARY

### Problems to be Solved by the Invention

The photolithography method typically has a baking process at a high temperature (for example, 230° C.). To form color filters on organic EL elements with low heat resistance, it is required to lower the baking temperature. Additionally, for light-shielding layers on micro displays that require finer patterns, further light-shielding properties and pattern shapes according to the design are demanded. The photosensitive resin composition described in Patent Document 1 had room for improvement in terms of light-shielding properties due to the inclusion of organic pigments. Furthermore, when attempting to enhance the light-shielding properties of the coating film by increasing the pigment ratio in the coating film, issues such as preservation stability as a photosensitive composition and residue generation after development arose.

The present disclosure aims to provide a photosensitive composition for organic EL display device that is excellent in preservation stability as a photosensitive composition, excellent in curability at a low temperature, has good developability, and is capable of forming a cured film having high light-shielding properties; and an organic EL display device including a cured film having high light-shielding properties.

### Means for Solving the Problems

A photosensitive composition for organic EL display device according to the present disclosure includes carbon black, a polymerizable compound, a photopolymerization initiator, and a photosensitive resin,
in which the photopolymerization initiator includes a compound having two or more oxime esters in one molecule,
the polymerizable compound includes a hexafunctional polymerizable compound and a trifunctional polymerizable compound, and
the photosensitive composition for organic EL display device includes 30% by mass to 50% by mass of the carbon black in a non-volatile content of the photosensitive composition for organic EL display device.

In one aspect of the photosensitive composition for organic EL display device, the compound having two or more oxime esters in one molecule includes a compound represented by the following chemical formula (1-1).

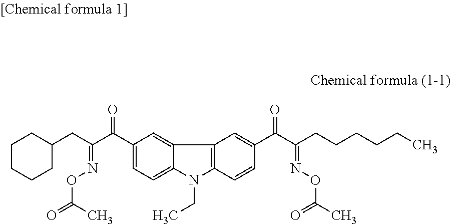 [SMILES: CCCCCC/C(=N\OC(C)=O)C(=O)c1ccc2c(c1)c1cc(C(=O)/C(CC3CCCCC3)=N/OC(C)=O)ccc1n2CC]

In one aspect of the photosensitive composition for organic EL display device, an average primary particle diameter of the carbon black is 50 nm or less.

In one aspect of the photosensitive composition for organic EL display device, a specific surface area of the carbon black is 80 m²/g to 260 m²/g.

One aspect of the photosensitive composition for organic EL display device includes 3% by mass to 16% by mass of the photopolymerization initiator in the non-volatile content of the photosensitive composition for organic EL display device.

In one aspect of the photosensitive composition for organic EL display device, a ratio of the photosensitive resin is 80% by mass or more in all resin components contained in the photosensitive composition.

An organic EL display device according to the present disclosure includes a light-shielding layer formed from the aforementioned photosensitive composition for organic EL display device.

### Effects of the Invention

According to the present disclosure, it is possible to provide a photosensitive composition for organic EL display device that is excellent in preservation stability as a photosensitive composition, excellent in curability at a low temperature, has good developability, and is capable of forming a cured film having high light-shielding properties; and an organic EL display device including a cured film having high light-shielding properties.

### BRIEF DESCRIPTION OF THE DRAWINGS

FIG. 1 is a schematic cross-sectional view of the organic EL display device.

### DESCRIPTION OF THE EMBODIMENTS

The following describes a photosensitive composition for organic EL display device, and an organic EL display device.

In the present disclosure, “to” used to indicate a numerical range is intended to include the numerical values described before and after the range as the lower limit and upper limit.

In the present disclosure, unless otherwise specified, the terms “(meth)acryloyl”, “(meth)acryl”, “(meth)acrylic acid”, “(meth)acrylate”, and “(meth)acrylamide” represent “acryloyl and/or methacryloyl”, “acryl and/or methacryl”, “acrylic acid and/or methacrylic acid”, “acrylate and/or methacrylate”, or “acrylamide and/or methacrylamide”, respectively.

### 1. Photosensitive Composition for Organic EL Display Device

The photosensitive composition for organic EL display device of the present disclosure (hereinafter also referred to simply as “photosensitive composition”) includes carbon black, a polymerizable compound, a photopolymerization initiator, and a photosensitive resin. The photopolymerization initiator includes a compound having two or more oxime esters in one molecule. The polymerizable compound includes a hexafunctional polymerizable compound and a trifunctional polymerizable compound. The photosensitive composition includes 30% by mass to 50% by mass of the carbon black in the non-volatile content of the photosensitive composition for organic EL display device.

The photosensitive composition having the above structure includes a compound having two or more oxime esters in one molecule as the photopolymerization initiator, resulting in excellent curability, especially at a low temperature. Therefore, even when forming a patterned cured film on an organic EL element by the photolithography method using this photosensitive composition, it is possible to form a light-shielding film without causing thermal damage to the organic EL element. In addition, because this photosensitive composition has excellent curability, this photosensitive composition exhibits superior developability even when the ratio of carbon black is 30% by mass to 50% by mass, enabling the formation of a cured film with the designed pattern shape and suppressing development residue. Furthermore, by increasing the ratio of carbon black, a cured film with high light-shielding properties can be formed.

This photosensitive composition includes at least carbon black, a polymerizable compound, a photopolymerization initiator, and a photosensitive resin, and may further include other components to the extent that enables this photosensitive composition to solve the problems of the present invention. The following describes each component that may be included in this photosensitive composition.

### <Carbon Black>

This photosensitive composition uses carbon black as the black component of the colorant. By using carbon black, a cured film with excellent light-shielding properties can be formed.

The average primary particle diameter of the carbon black is preferably 50 nm or less, more preferably 10 nm to 30 nm, and even more preferably 15 nm to 25 nm, in terms of improved dispersibility and light-shielding properties, and good fine line patterns (linearity). By setting the average primary particle diameter to 10 nm or more, development residue can also be suppressed. In addition, the specific surface area of the carbon black is preferably 80 m²/g to 260 m²/g, more preferably 100 m²/g to 180 m²/g, and even more preferably 110 m²/g to 120 m²/g, in terms of improved dispersibility and light-shielding properties. By setting the specific surface area to 80 m²/g or more, adhesion to the base material (organic EL element) is also improved.

The primary particle diameter of the carbon black is performed by directly measuring the size of the primary particles from electron microscope images obtained using TEM (transmission electron microscope). Specifically, the short axis diameter and long axis diameter of individual carbon black primary particles are measured, and the average thereof is taken as the particle diameter. Then, for 20 or more carbon black particles, the volume of each particle is approximated as a cube of the particle diameter, and the volume average particle diameter is taken as the average primary particle diameter.

The aforementioned carbon black may be a commercially available product. The commercially available products include #40, #44, #45, #45L, #47, and #52 manufactured by Mitsubishi Chemical, Printex55 manufactured by Orion Engineered Carbons, and the like. The carbon black may be used alone or in a mixture of two or more types.

The aforementioned carbon black may be subjected to a fine-grinding treatment. The fine-grinding method can be appropriately selected from known methods such as wet grinding, dry grinding, and dissolution-precipitation method, and as for wet grinding, a salt milling treatment by a kneader method can be mentioned.

The salt milling treatment is a process in which a mixture of carbon black, water-soluble inorganic salt, and water-soluble organic solvent is mechanically kneaded while heating using a kneading machine such as a kneader, a two-roll mill, a three-roll mill, a ball mill, an attritor, or a sand mill, and then the water-soluble inorganic salt and water-soluble organic solvent are removed by washing with water. The water-soluble inorganic salt (for example, sodium chloride) acts as a grinding aid, and during salt milling, the carbon black is ground utilizing the high hardness of the inorganic salt. By optimizing the conditions for the salt milling treatment of the carbon black, it is possible to obtain carbon black with a very fine primary particle diameter, a narrow distribution width, and a sharp particle size distribution.

When performing the salt milling treatment on the carbon black, a resin may be added as required. The type of the resin used is not particularly limited, and a natural resin, a modified natural resin, a synthetic resin, and a synthetic resin modified with a natural resin can be used. The resin used is preferably solid at room temperature and water-insoluble, and it is further preferable that the resin is partially soluble in the aforementioned organic solvent. The amount of the resin used is preferably in the range of 5 parts by mass to 200 parts by mass with respect to 100 parts by mass of the carbon black.

By performing the salt milling treatment, the average primary particle diameter of the carbon black can be adjusted to the aforementioned range.

The content ratio of the carbon black in this photosensitive composition is 30% by mass to 50% by mass in the non-volatile content of this photosensitive composition. By setting the content ratio to 30% by mass to 50% by mass, both the preservation stability of the photosensitive composition and the light-shielding properties of the cured product can be achieved.

### (Other Colorants)

This photosensitive composition may include other colorants besides the carbon black to the extent that the effects of the present invention are achieved. Other colorants include known black pigments and organic pigments of various colors. The content ratio of other colorants in this photosensitive composition is preferably 5 parts by mass or less, more preferably 1 part by mass or less, and even more preferably 0.1 parts by mass or less, with respect to 100 parts by mass of the carbon black, in terms of preservation stability of the composition and light-shielding properties.

### <Polymerizable Compound>

The polymerizable compound is appropriately selected and used from compounds that are polymerizable by the action of the photopolymerization initiator which will be described later. As the polymerizable compound, a compound having one or more ethylenically unsaturated bonds in one molecule is preferable, and a multifunctional compound having two or more ethylenically unsaturated bonds is preferable. Further, examples of ethylenically unsaturated bonds include vinyl groups and (meth)acryloyl groups, with (meth)acryloyl groups being preferable, and acryloyl groups being preferable. The polymerizable compound also includes a polymerizable oligomer with a molecular weight of about 1000 or less.

Examples of the polymerizable compound include di(meth)acrylates of alkylene glycols such as ethylene glycol and propylene glycol; di(meth)acrylates of polyalkylene glycols such as polyethylene glycol and polypropylene glycol; poly(meth)acrylates of polyhydric alcohols having three or more valences such as glycerin, trimethylolpropane, pentaerythritol, and dipentaerythritol; oligo(meth)acrylates of polyester, epoxy resin, urethane resin, alkyd resin, silicone resin, spirane resin, and the like.

This photosensitive composition preferably includes a hexafunctional polymerizable compound in terms of increased curability. Examples of the hexafunctional polymerizable compound include dipentaerythritol hexa(meth)acrylate and the like.

Furthermore, it is more preferable for this photosensitive composition to use a combination of a hexafunctional polymerizable compound and a trifunctional polymerizable compound. By using a trifunctional polymerizable compound in combination, the linearity of the patterned cured film is improved. The weight ratio of the hexafunctional polymerizable compound to the trifunctional polymerizable compound is preferably 99/1 to 1/99, and more preferably 90/10 to 50/50.

Specific examples of the trifunctional polymerizable compound include trimethylolpropane tri(meth)acrylate, trimethylolpropane EO-modified tri(meth)acrylate, trimethylolpropane PO-modified tri(meth)acrylate, pentaerythritol tri(meth)acrylate, and the like. Among these, alkylene oxide-modified tri(meth)acrylates such as trimethylolpropane EO-modified tri(meth)acrylate and trimethylolpropane PO-modified tri(meth)acrylate are preferable in terms of improved developability. These may be used in combination of two or more types.

The aforementioned polymerizable compound may further use other (meth)acrylate monomers. Specifically, examples include, but are not limited to, methyl (meth)acrylate, ethyl (meth)acrylate, 2-hydroxyethyl (meth)acrylate, 2-hydroxypropyl (meth)acrylate, cyclohexyl (meth)acrylate, β-carboxyethyl (meth)acrylate, polyethylene glycol di(meth)acrylate, 1,6-hexanediol di(meth)acrylate, triethylene glycol di(meth)acrylate, tripropylene glycol di(meth)acrylate, pentaerythritol tetra(meth)acrylate, 1,6-hexanediol diglycidyl ether di(meth)acrylate, bisphenol A diglycidyl ether di(meth)acrylate, neopentyl glycol diglycidyl ether di(meth)acrylate, dipentaerythritol penta(meth)acrylate, tricyclodecanyl (meth)acrylate, (meth)acrylic acid ester of methylolated melamine, various acrylic acid esters and methacrylic acid esters such as epoxy (meth)acrylate, (meth)acrylic acid, styrene, vinyl acetate, hydroxyethyl vinyl ether, ethylene glycol divinyl ether, pentaerythritol trivinyl ether, (meth)acrylamide, N-hydroxymethyl (meth)acrylamide, N-vinylformamide, acrylonitrile, and the like.

The content ratio of the polymerizable compound in the photosensitive composition is preferably 5% by mass to 40% by mass, and more preferably 20% by mass to 30% by mass in the non-volatile content of this photosensitive composition. By setting the content ratio within the aforementioned range, it is possible to form fine patterns without peeling, suppress the elongation of the tapered portion of the pattern, and form high-definition fine pixel patterns.

### <Photopolymerization Initiator>

The photopolymerization initiator can be appropriately selected and used from initiators that promote the polymerization reaction of the polymerizable compound by the action of light.

In this photosensitive composition, it is preferable that the photopolymerization initiator includes a compound having two or more oxime esters (>C═N—OC(═O)—) in one molecule (hereinafter also referred to as an oxime ester-based photopolymerization initiator) in terms of excellent ultraviolet curability, color characteristics, chemical resistance, and developability.

The oxime ester-based photopolymerization initiator undergoes cleavage of the N—O bond of the oxime by absorbing ultraviolet, and generates iminyl radicals and alkyloxy radicals. These radicals further decompose to generate highly active radicals, allowing pattern formation with a small exposure amount. Additionally, the oxime ester-based photopolymerization initiator has high quantum efficiency, which provides excellent curability even in the case where the colorant (carbon black) in the composition is highly concentrated. This photosensitive composition enables the formation of a cured film with fine patterns that have excellent developability and good patterning properties through the photolithography method. The oxime ester-based photopolymerization initiator also provides superior light-shielding properties and chemical resistance to the cured film.

Among compounds having two or more oxime esters (>C═N—OC(═O)—) in one molecule, the oxime ester-based photopolymerization initiator represented by the following general formula (1) is preferable.

### (Oxime Ester-Based Photopolymerization Initiator Represented by General Formula (1))

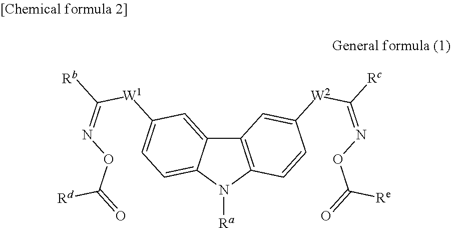

(In general formula (1), W¹ and W² each independently represent a carbonyl bond (—CO—) or a single bond, and at least one of W¹ and W² is a carbonyl bond (—CO—).

R^{a} is an alkyl group having 2 to 6 carbon atoms, R^{b} is an alkyl group having 4 to 10 carbon atoms, R^{c} is a group including at least a hydrocarbon ring or a heterocyclic ring, and may further include at least one divalent linking group selected from an alkylene chain having 1 to 4 carbon atoms, a thioether bond (—S—), an ether bond (—O—), and a carbonyl bond (—CO—), and R^{b} and R^{c} are different substituents from each other. R^{d} and R^{e} are each independently an alkyl group having 1 to 6 carbon atoms.)

For the alkyl group in R^{a}, examples include straight-chain alkyl groups such as ethyl group, propyl group, butyl group, hexyl group, and the like.

For the alkyl group in R^{b}, examples include straight-chain alkyl groups such as butyl group, hexyl group, octyl group, dodecyl group, and the like.

For the hydrocarbon ring in R^{c}, examples include aliphatic hydrocarbon rings such as cyclohexyl group, aromatic hydrocarbon rings such as phenyl group, naphthyl group, anthracenyl group, and the like. Additionally, for the heterocyclic ring in R^{c}, examples include rings in which one or two or more carbon atoms of the aforementioned hydrocarbon rings are replaced with nitrogen atoms, oxygen atoms, or sulfur atoms.

Furthermore, for the alkyl groups in R^{d} and R^{e}, examples include straight-chain alkyl groups such as methyl group, ethyl group, propyl group, butyl group, hexyl group, and the like.

Among the oxime ester-based photopolymerization initiators represented by general formula (1), the compound represented by the following chemical formula (1-1) is preferable.

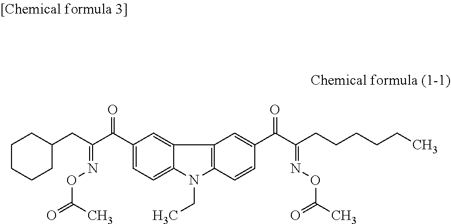

The photopolymerization initiator may also be other photopolymerization initiators used together with the compound having two or more oxime esters in one molecule as mentioned above. Examples of other photopolymerization initiators include compounds having one oxime ester in one molecule, or photopolymerization initiators other than the oxime ester-based polymerization initiator. Among the oxime ester-based photopolymerization initiators having one oxime ester in one molecule, the oxime ester-based photopolymerization initiator represented by the following general formula (2) or (3) is preferable.

### (Oxime Ester-Based Photopolymerization Initiator Represented by General Formula (2))

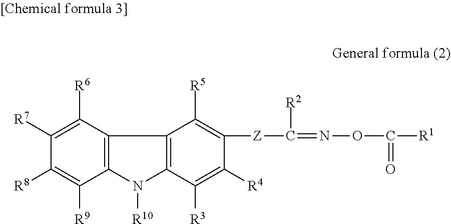

(In general formula (2), Z represents a direct bond or a —C(═O)— group, R¹ represents an alkyl group having 1 to 20 carbon atoms which may have a substituent, R² represents an alkyl group having 1 to 20 carbon atoms which may have a substituent, or an aryl group which may have a substituent, R³ to R¹⁰ each independently represent a hydrogen atom, an alkyl group having 1 to 20 carbon atoms which may have a substituent, an aryl group which may have a substituent, a nitro group, or an R¹¹—C(═O)— group. R¹¹ represents an aryl group which may have a substituent.)

For the alkyl group having 1 to 20 carbon atoms in R¹ to R¹⁰, examples include straight-chain alkyl groups such as methyl group, ethyl group, propyl group, butyl group, hexyl group, octyl group, dodecyl group, and the like.

For the aryl group in R² to R¹¹, examples include phenyl group, naphthyl group, anthracenyl group, and the like.

When Z is a direct bond, it indicates that Z does not have any atom, and the two atoms connected to Z in general formula (2) are directly bonded to each other.

The substituents that the aforementioned alkyl group and aryl group may have indicate that they may have substituents in place of the hydrogen atoms possessed by the alkyl group or aryl group. Such substituents include, for example, halogen atoms such as fluorine atom, chlorine atom, bromine atom, and iodine atom; alkoxy groups such as methoxy group, ethoxy group, and tert-butoxy group; aryloxy groups such as phenoxy group and p-tolyloxy group; alkoxycarbonyl groups such as methoxycarbonyl group, butoxycarbonyl group, and phenoxycarbonyl group; acyloxy groups such as acetoxy group, propionyloxy group, and benzoyloxy group; acyl groups such as acetyl group, benzoyl group, isobutyryl group, acryloyl group, methacryloyl group, and methoxalyl group; alkylsulfanyl groups such as methylsulfanyl group and tert-butylsulfanyl group; arylsulfanyl groups such as phenylsulfanyl group and p-tolylsulfanyl group; alkylamino groups such as methylamino group and cyclohexylamino group; dialkylamino groups such as dimethylamino group, diethylamino group, morpholino group, and piperidino group; arylamino groups such as phenylamino group and p-tolylamino group; alkyl groups such as methyl group, ethyl group, tert-butyl group, dodecyl group, cyclopropyl group, cyclobutyl group, cyclopentyl group, cyclohexyl group, cycloheptyl group, cyclooctyl group, and cyclooctadecyl group; aryl groups such as phenyl group, p-tolyl group, xylyl group, cumenyl group, naphthyl group, anthryl group, and phenanthryl group; heterocyclic groups such as furyl group and thienyl group; as well as hydroxyl group, carboxyl group, formyl group, mercapto group, sulfo group, mesyl group, p-toluenesulfonyl group, amino group, nitro group, cyano group, trifluoromethyl group, trichloromethyl group, trimethylsilyl group, phosphinico group, phosphono group, trimethylammoniumyl group, dimethylsulfoniumyl group, triphenylphenacylphosphoniumyl group, and the like.

Moreover, one or more of these substituents or one or more types can exist, and the hydrogen atoms of these substituents may be further substituted with other substituents.

The oxime ester-based photopolymerization initiator represented by general formula (2) is preferably one in which Z is a direct bond or a —C(═O)— group, R¹ is an alkyl group having 1 to 20 carbon atoms which may have a substituent, R² is an alkyl group having 1 to 20 carbon atoms which may have a substituent or an aryl group which may have a substituent, and R³ to R¹⁰ are each independently a hydrogen atom, an alkyl group having 1 to 20 carbon atoms which may have a substituent, an aryl group which may have a substituent, a nitro group, or an R¹¹—CO— group. Among these, it is more preferable that R⁴ to R⁶ and R⁸ to R¹⁰ are hydrogen atoms, R⁷ is a hydrogen atom or an R¹¹—CO— group, and R¹¹ is an aryl group which may have a substituent.

Among the oxime ester-based photopolymerization initiators represented by general formula (2), the compound represented by the following chemical formula (2-1) or (2-2) is preferable.

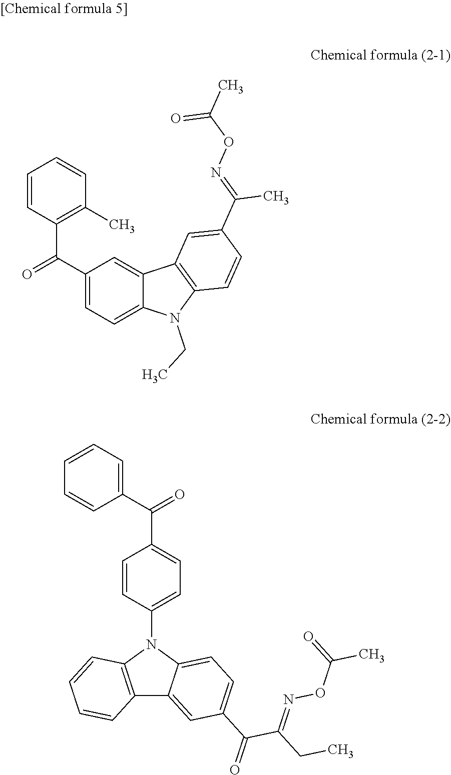

### (Oxime Ester-Based Photopolymerization Initiator Represented by General Formula (3))

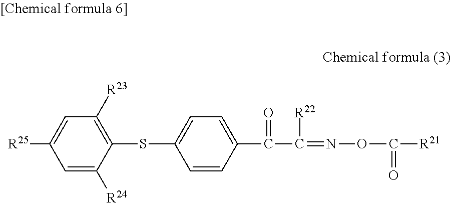

(In general formula (3), R²¹ and R²² are each independently a hydrogen atom, an alkyl group having 1 to 20 carbon atoms which may have a substituent, or an aryl group which may have a substituent; R²³ and R²⁴ are each independently a hydrogen atom, an alkyl group having 1 to 20 carbon atoms which may have a substituent, or an aryl group which may have a substituent; R²⁵ is a hydrogen atom, an alkyl group having 1 to 20 carbon atoms which may have a substituent, an aryl group which may have a substituent, or an R²⁶—CO— group; and R²⁶ is an alkyl group having 1 to 20 carbon atoms which may have a substituent, an aryl group which may have a substituent, or a heterocyclic group.)

The alkyl groups having 1 to 20 carbon atoms in R²¹ to R²⁶ can be the same as the alkyl groups having 1 to 20 carbon atoms in the aforementioned R¹ to R¹⁰.

The aryl groups in R²¹ to R²⁶ can be the same as the aryl groups in the aforementioned R² to R¹¹.

For the heterocyclic ring in the heterocyclic group of R²⁶, examples include furan, thiophene, pyrrole, oxazole, thiazole, imidazole, pyrazole, pyran, pyrone, pyridine, pyrone, pyridazine, pyrimidine, pyrazine, benzofuran, thionaphthene, indole, carbazole, coumarin, quinoline, phthalazine, quinoxaline, and the like.

In addition, the substituents that the alkyl group and the aryl group may have can be the same as the substituent in the aforementioned general formula (2).

In the oxime ester-based photopolymerization initiator represented by general formula (3), it is preferable that R²¹ is an aryl group which may have a substituent, R²² is an alkyl group having 1 to 20 carbon atoms which may have a substituent, R²³ and R²⁴ are hydrogen atoms, and R²⁵ is a hydrogen atom or an R²⁶—CO— group.

Among the oxime ester-based photopolymerization initiators represented by general formula (3), the compound represented by the following chemical formula (3-1) is preferable.

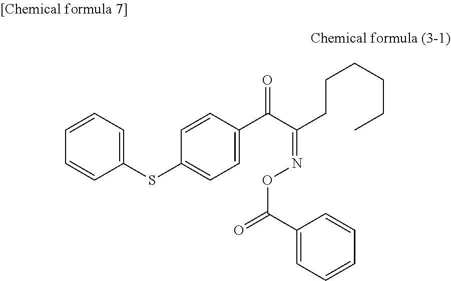

In addition to the aforementioned oxime ester-based photopolymerization initiator, examples of other photopolymerization initiators include acetophenone-based compounds such as 4-phenoxydichloroacetophenone, 4-t-butyl-dichloroacetophenone, diethoxyacetophenone, 1-(4-isopropylphenyl)-2-hydroxy-2-methylpropan-1-one, 1-hydroxycyclohexylphenylketone, 2-methyl-1-[4-(methylthio)phenyl]-2-morpholinopropan-1-one, 2-(dimethylamino)-2-[(4-methylphenyl)methyl]-1-[4-(4-morpholinyl)phenyl]-1-butanone, and 2-benzyl-2-dimethylamino-1-(4-morpholinophenyl)-butan-1-one; benzoin-based compounds such as benzoin, benzoin methyl ether, benzoin ethyl ether, benzoin isopropyl ether, and benzyl dimethyl ketal; benzophenone-based compounds such as benzophenone, benzoyl benzoic acid, methyl benzoyl benzoate, 4-phenylbenzophenone, hydroxybenzophenone, acrylated benzophenone, 4-benzoyl-4′-methyldiphenyl sulfide, and 3,3′,4,4′-tetra(t-butylperoxycarbonyl)benzophenone; thioxanthone-based compounds such as thioxanthone, 2-chlorothioxanthone, 2-methylthioxanthone, isopropylthioxanthone, 2,4-diisopropylthioxanthone, and 2,4-diethylthioxanthone; triazine-based compounds such as 2,4,6-trichloro-s-triazine, 2-phenyl-4,6-bis(trichloromethyl)-s-triazine, 2-(p-methoxyphenyl)-4,6-bis(trichloromethyl)-s-triazine, 2-(p-tolyl)-4,6-bis(trichloromethyl)-s-triazine, 2-piperonyl-4,6-bis(trichloromethyl)-s-triazine, 2,4-bis(trichloromethyl)-6-styryl-s-triazine, 2-(naphth-1-yl)-4,6-bis(trichloromethyl)-s-triazine, 2-(4-methoxy-naphth-1-yl)-4,6-bis(trichloromethyl)-s-triazine, 2,4-trichloromethyl-(piperonyl)-6-triazine, and 2,4-trichloromethyl-(4′-methoxystyryl)-6-triazine; phosphine-based compounds such as bis(2,4,6-trimethylbenzoyl)phenylphosphine oxide and 2,4,6-trimethylbenzoyldiphenylphosphine oxide; quinone-based compounds such as 9,10-phenanthrenequinone, camphorquinone, and ethylanthraquinone; borate-based compounds; carbazole-based compounds; imidazole-based compounds; titanocene-based compounds, and the like.

These photopolymerization initiators can be used individually or, as required, in a mixture of two or more types in any ratio.

As commercially available photopolymerization initiators, acetophenone-based compounds include, all manufactured by BASF Japan, “IRGACURE 907” (2-methyl-1-[4-(methylthio)phenyl]-2-morpholinopropan-1-one), “IRGACURE 369” (2-(dimethylamino)-2-[(4-methylphenyl)methyl]-1-[4-(4-morpholinyl)phenyl]-1-butanone), and “IRGACURE 379” 2-benzyl-2-dimethylamino-1-(4-morpholinophenyl)-butan-1-one; and phosphine-based compounds include, all manufactured by BASF Japan, “IRGACURE 819” (bis(2,4,6-trimethylbenzoyl)phenylphosphine oxide), “IRGACURE TPO” (2,4,6-trimethylbenzoyl diphenylphosphine oxide), and the like.

The content ratio of the photopolymerization initiator in the photosensitive composition is preferably 3% by mass to 16% by mass in the non-volatile content of this photosensitive composition, and more preferably 10% by mass to 16% by mass from the viewpoint of photocurability and developability. If the photopolymerization initiator is 3 parts by mass or more, adhesion to the base material is excellent. In addition, if the photopolymerization initiator is 16 parts by mass or less, excellent resolution is exhibited.

Moreover, in the photopolymerization initiator, the ratio of the compound having two or more oxime esters in one molecule is preferably 80% by mass or more of the total amount of the photopolymerization initiator, and more preferably 90% by mass or more.

### <Sensitizer>

This photosensitive composition may further include a sensitizer.

Examples of the sensitizer include unsaturated ketones such as chalcone derivatives and dibenzylideneacetone; 1,2-diketone derivatives such as benzil and camphorquinone; polymethine dyes such as benzoin derivatives, fluorene derivatives, naphthoquinone derivatives, anthraquinone derivatives, xanthene derivatives, thioxanthene derivatives, xanthone derivatives, thioxanthone derivatives, coumarin derivatives, ketocoumarin derivatives, cyanine derivatives, merocyanine derivatives, and oxonol derivatives; acridine derivatives, azine derivatives, thiazine derivatives, oxazine derivatives, indoline derivatives, azulene derivatives, azulenium derivatives, squarylium derivatives, porphyrin derivatives, tetraphenylporphyrin derivatives, triarylmethane derivatives, tetrabenzoporphyrin derivatives, tetrapyrazinoporphyrazine derivatives, phthalocyanine derivatives, tetraazaporphyrazine derivatives, tetraquinoxalinoporphyrazine derivatives, naphthalocyanine derivatives, subphthalocyanine derivatives, pyrylium derivatives, thiopyrylium derivatives, tetraphyrin derivatives, annulene derivatives, spiropyran derivatives, spirooxazine derivatives, thiospiropyran derivatives, metal arene complexes, organic ruthenium complexes, or Michler's ketone derivatives, α-acyloxyesters, acylphosphine oxides, methyl phenylglyoxylate, benzil, 9,10-phenanthrenequinone, camphorquinone, ethyl anthraquinone, 4,4′-diethylisophthalophenone, 3,3′ or 4,4′-tetra(t-butylperoxycarbonyl)benzophenone, 4,4′-bis(diethylamino)benzophenone, and the like.

Among the aforementioned sensitizers, particularly suitable sensitizers for sensitization include thioxanthone derivatives, Michler's ketone derivatives, and carbazole derivatives. More specifically, 2,4-diethylthioxanthone, 2-chlorothioxanthone, 2,4-dichlorothioxanthone, 2-isopropylthioxanthone, 4-isopropylthioxanthone, 1-chloro-4-propoxythioxanthone, 4,4′-bis(dimethylamino)benzophenone, 4,4′-bis(diethylamino)benzophenone, 4,4′-bis(ethylmethylamino)benzophenone, N-ethylcarbazole, 3-benzoyl-N-ethylcarbazole, 3,6-dibenzoyl-N-ethylcarbazole, and the like can be mentioned.

Moreover, examples of commercially available sensitizers include “KAYACURE DETX-S” (2,3-diethylthioxanthone manufactured by Nippon Kayaku Co., Ltd.), “EAB-F” (4,4′-bis(diethylamino)benzophenone manufactured by Hodogaya Chemical Co., Ltd.), and the like.

In addition, sensitizers that exhibit absorption to light in the range from ultraviolet to near-infrared can also be included.

The sensitizers can be used individually or, as required, in a mixture of two or more types in any ratio.

In the case of using a sensitizer, the content is preferably 3 parts by mass to 60 parts by mass with respect to 100 parts by mass of the photopolymerization initiator included in the photosensitive composition, and more preferably 5 parts by mass to 50 parts by mass from the viewpoint of photocurability and developability.

### <Resin>

The photosensitive composition includes a resin, and the resin includes at least a photosensitive resin. The resin is preferably a transparent resin that has a transmittance of 80% or more in the entire wavelength range of 400 nm to 700 nm when forming a film with a thickness of 2 μm, and the transmittance is preferably 95% or more. The resin includes, for example, a binder resin and a resin-type dispersant. Examples of the binder resin include a thermoplastic resin, an alkali-soluble resin, an alkali-soluble photosensitive resin, and the like. In terms of patterning properties by the photolithography method, it is preferable that the resin includes an alkali-soluble resin. The photosensitive resin is a resin having polymerizable unsaturated groups such as vinyl group, (meth)acryloyl group, (meth)allyl group, and the like as photosensitive groups. The photosensitive resin is a resin having photosensitive groups among the binder resin and the resin-type dispersant. In this photosensitive composition, the resin refers to a resin having a molecular weight or weight average molecular weight of 1000 or more, and includes a resin having various functionalities which will be described later. First, the binder resin will be described.

### (Alkali-Soluble Resin)

The alkali-soluble resin includes, for example, resins having acidic groups such as carboxyl group, sulfo group, and the like. The thermoplastic resin having alkali solubility includes, for example, acrylic resin having acidic groups, α-olefin/(anhydrous) maleic acid copolymer, styrene/styrenesulfonic acid copolymer, ethylene/(meth)acrylic acid copolymer, or isobutylene/(anhydrous) maleic acid copolymer, and the like. Among these, acrylic resin having acidic groups and styrene/styrenesulfonic acid copolymer are preferable in terms of improved developability, heat resistance, and transparency.

### (Thermoplastic Resin)

The thermoplastic resin may include resins that do not have alkali solubility. Thermoplastic resins without alkali solubility include, for example, acrylic resin, butyral resin, styrene-maleic acid copolymer, chlorinated polyethylene, chlorinated polypropylene, polyvinyl chloride, vinyl chloride-vinyl acetate copolymer, polyvinyl acetate, polyurethane-based resin, polyester resin, vinyl-based resin, alkyd resin, polystyrene resin, polyamide resin, rubber-based resin, cyclized rubber-based resin, celluloses, polyethylene (HDPE, LDPE), polybutadiene, and polyimide resin.

### (Alkali-Soluble Photosensitive Resin)

The alkali-soluble photosensitive resin has photosensitivity due to having polymerizable unsaturated groups. The alkali-soluble photosensitive resin may have alkali solubility and photosensitivity, and while known resins can be used, resins synthesized by the following methods (i) and (ii) are preferable. When the alkali-soluble photosensitive resin is used, three-dimensional crosslinking occurs upon light irradiation, which increases the crosslinking density, thereby improving the chemical resistance of the film.

[Method (i)]

Method (i) includes, for example, first synthesizing a polymer of an epoxy group-containing monomer and other monomers. Subsequently, a monocarboxyl group-containing monomer is added to the epoxy group of the aforementioned polymer, and then a polybasic acid anhydride is reacted with the resulting hydroxyl group to obtain an alkali-soluble photosensitive resin. The monocarboxyl group-containing monomer is a monomer having one carboxyl group.

Examples of the epoxy group-containing monomer include glycidyl (meth)acrylate, methyl glycidyl (meth)acrylate, 2-glycidoxyethyl (meth)acrylate, 3,4-epoxybutyl (meth)acrylate, and 3,4-epoxycyclohexyl (meth)acrylate. Among these, glycidyl (meth)acrylate is preferable from the viewpoint of reactivity.

Examples of the monocarboxyl group-containing monomer include monocarboxylic acids such as (meth)acrylic acid, crotonic acid, o-, m-, p-vinylbenzoic acid, α-position haloalkyl, alkoxyl, halogen, nitro, cyano substituted products of (meth)acrylic acid, and the like.

Examples of the polybasic acid anhydride include tetrahydrophthalic anhydride, phthalic anhydride, hexahydrophthalic anhydride, succinic anhydride, maleic anhydride, and the like. The polybasic acid anhydride may have carboxyl groups that do not form acid anhydrides.

Examples of other monomers include (meth)acrylates such as methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, 2-ethylhexyl (meth)acrylate, cyclohexyl (meth)acrylate, stearyl (meth)acrylate, lauryl (meth)acrylate, tetrahydrofurfuryl (meth)acrylate, isobornyl (meth)acrylate, phenyl (meth)acrylate, benzyl (meth)acrylate, phenoxyethyl (meth)acrylate, phenoxydiethylene glycol (meth)acrylate, methoxy polypropylene glycol (meth)acrylate, or ethoxy polyethylene glycol (meth)acrylate; (meth)acrylamides such as (meth)acrylamide, N,N-dimethyl (meth)acrylamide, N,N-diethyl (meth)acrylamide, N-isopropyl (meth)acrylamide, diacetone (meth)acrylamide, or acryloyl morpholine; styrenes such as styrene or α-methylstyrene; vinyl ethers such as ethyl vinyl ether, n-propyl vinyl ether, isopropyl vinyl ether, n-butyl vinyl ether, or isobutyl vinyl ether; fatty acid vinyl esters such as vinyl acetate or vinyl propionate, and the like.

Additionally, N-substituted maleimides such as cyclohexyl maleimide, phenyl maleimide, methyl maleimide, ethyl maleimide, 1,2-bismaleimidoethane, 1,6-bismaleimidohexane, 3-maleimidopropionic acid, 6,7-methylenedioxy-4-methyl-3-maleimidocoumarin, 4,4′-bismaleimidodiphenylmethane, bis(3-ethyl-5-methyl-4-maleimidophenyl)methane, N,N′-1,3-phenylenedimaleimide, N,N′-1,4-phenylenedimaleimide, N-(1-pyrenyl)maleimide, N-(2,4,6-trichlorophenyl)maleimide, N-(4-aminophenyl)maleimide, N-(4-nitrophenyl)maleimide, N-benzylmaleimide, N-bromomethyl-2,3-dichloromaleimide, N-succinimidyl-3-maleimidobenzoate, N-succinimidyl-3-maleimidopropionate, N-succinimidyl-4-maleimidobutyrate, N-succinimidyl-6-maleimidohexanoate, N-[4-(2-benzoimidazolyl)phenyl]maleimide, and 9-maleimidoacridine; EO-modified cresol acrylate, n-nonylphenoxy polyethylene glycol acrylate, phenoxyethyl acrylate, ethoxylated phenyl acrylate, ethylene oxide (EO)-modified (meth)acrylate of phenol, EO or propylene oxide (PO)-modified (meth)acrylate of p-cumylphenol, EO-modified (meth)acrylate of nonylphenol, PO-modified (meth)acrylate of nonylphenol, and the like can be mentioned.

[Method (ii)]

Method (ii) includes, for example, synthesizing a hydroxyl group-containing monomer, a carboxyl group-containing monomer, and other monomers to prepare a polymer. Subsequently, the isocyanate groups of an isocyanate group-containing monomer are reacted with the hydroxyl groups of the aforementioned polymer to synthesize an alkali-soluble photosensitive resin.

Examples of the hydroxyl group-containing monomer include hydroxyalkyl methacrylates such as 2-hydroxyethyl (meth)acrylate, 2- or 3-hydroxypropyl (meth)acrylate, 2- or 3- or 4-hydroxybutyl (meth)acrylate, glycerol mono(meth)acrylate, or cyclohexanedimethanol mono(meth)acrylate. Additionally, polyether mono(meth)acrylates obtained by addition polymerization of ethylene oxide, propylene oxide, and/or butylene oxide to hydroxyalkyl (meth)acrylates, and polyester mono(meth)acrylates obtained by adding poly-γ-valerolactone, poly-ε-caprolactone, and/or poly-12-hydroxystearic acid can also be mentioned. Among these, 2-hydroxyethyl methacrylate and glycerol mono(meth)acrylate are preferable, with glycerol mono(meth)acrylate being more preferable.

Examples of the isocyanate group-containing monomer include 2-(meth)acryloylethyl isocyanate, 2-(meth)acryloyloxyethyl isocyanate, or 1,1-bis[methacryloyloxy]ethyl isocyanate.

In addition to the monomers mentioned above, other monomers that can be used include the other monomers exemplified in the aforementioned method (i), as well as phosphate ester group-containing monomers.

The phosphate ester group-containing monomers are, for example, compounds obtained by reacting phosphate esterification agents such as phosphorus pentoxide or polyphosphoric acid with the hydroxyl groups of the hydroxyl group-containing monomer.

### (Resin-Type Dispersant)

The resin-type dispersant has a colorant-affinity site that adsorbs to carbon black and a relaxation site that has high affinity with components other than carbon black and causes steric repulsion between dispersed particles.

Examples of the resin-type dispersant with acidic groups include urethane-based dispersants such as polyurethanes, polycarboxylic acid esters such as polyacrylates, unsaturated polyamides, polycarboxylic acids, polycarboxylic acid (partial) amine salts, polycarboxylic acid ammonium salts, polycarboxylic acid alkylamine salts, polysiloxanes, long-chain polyaminoamide phosphates, hydroxyl group-containing polycarboxylic acid esters, and modified products of these; amides and salts thereof formed by the reaction of poly(lower alkylene imine) with polyesters having free carboxyl groups; (meth)acrylic acid-styrene copolymers, (meth)acrylic acid-(meth)acrylic acid ester copolymers, styrene-maleic acid copolymers, polyvinyl alcohol, polyvinylpyrrolidone; polyesters, modified polyacrylates, ethylene oxide/propylene oxide adducts, phosphate ester-based compounds, and the like.

Examples of polymeric dispersants with basic groups include nitrogen atom-containing graft copolymers, nitrogen atom-containing acrylic block copolymers and urethane-based polymeric dispersants having functional groups including tertiary amino groups, quaternary ammonium bases, or nitrogen-containing heterocyclic rings in the side chains, and the like.

It is preferable that the resin-type dispersant has photosensitivity. Additionally, it is preferable that the resin-type dispersant has acidic groups, with an acid value preferably in the range of 5 mgKOH/g to 150 mgKOH/g, more preferably 10 mgKOH/g to 120 mgKOH/g, and even more preferably 20 mgKOH/g to 120 mgKOH/g. Furthermore, it is preferable that the resin-type dispersant does not have amines, with an amine value of preferably 10 mgKOH/g or less, more preferably 5 mgKOH/g or less, and even more preferably 1 mgKOH/g or less.

Examples of commercially available resin-type dispersants include Disperbyk-101, 103, 107, 108, 110, 111, 116, 130, 140, 154, 161, 162, 163, 164, 165, 166, 170, 171, 174, 180, 181, 182, 183, 184, 185, 190, 2000, 2001, 2020, 2025, 2050, 2070, 2095, 2150, 2155, or Anti-Terra-U, 203, 204, or BYK-P104, P104S, 220S, 6919, or Lactimon, Lactimon-WS, or Bykumen, etc. manufactured by BYK Chemie Japan; SOLSPERSE-3000, 9000, 13000, 13240, 13650, 13940, 16000, 17000, 18000, 20000, 21000, 24000, 26000, 27000, 28000, 31845, 32000, 32500, 32550, 33500, 32600, 34750, 35100, 36600, 38500, 41000, 41090, 53095, 55000, 76500, etc. manufactured by Lubrizol Japan; EFKA-46, 47, 48, 452, 4008, 4009, 4010, 4015, 4020, 4047, 4050, 4055, 4060, 4080, 4400, 4401, 4402, 4403, 4406, 4408, 4300, 4310, 4320, 4330, 4340, 450, 451, 453, 4540, 4550, 4560, 4800, 5010, 5065, 5066, 5070, 7500, 7554, 1101, 120, 150, 1501, 1502, 1503, etc. manufactured by BASF Japan; Ajisper PA111, PB711, PB821, PB822, PB824, etc. manufactured by Ajinomoto Fine-Techno, and the like.

### (Resin Having Thermosetting Groups)

Additionally, the aforementioned resins may have thermosetting groups. Examples of the thermosetting groups include epoxy groups, oxetanyl groups, and the like.

The weight average molecular weight (Mw) of the resin is preferably 2,000 to 40,000, more preferably 3,000 to 30,000, and even more preferably 4,000 to 20,000. The value of Mw/Mn is preferably 10 or less. Mn is the number average molecular weight.

The acid value of the resin is preferably 50 mgKOH/g to 200 mgKOH/g, more preferably 70 mgKOH/g to 180 mgKOH/g, and even more preferably 90 mgKOH/g to 170 mgKOH/g. An appropriate acid value can achieve a high-level balance of alkali solubility, adhesion, and residue suppression.

The resin can be used alone or in combination of two or more types. The content of the resin is preferably 20 parts by mass to 400 parts by mass, and more preferably 50 parts by mass to 250 parts by mass, with respect to 100 parts by mass of carbon black. Including an appropriate amount facilitates film formation, and tends to provide good light-shielding properties. In terms of good film-forming properties and various resistances, 20 parts by mass or more is preferable, and 400 parts by mass or less is preferable from the viewpoint of obtaining good light-shielding properties as the carbon black concentration becomes relatively high.

Additionally, in the total resin components included in this photosensitive composition, the ratio of the photosensitive resin is preferably 80% by mass or more, and more preferably 90% by mass or more. By including a large amount of the photosensitive resin, the developability and curability are further improved. Among these, it is preferable that the resin-type dispersant described later has photosensitivity. The “total resin components” refers to the mass ratio of the photosensitive resin in the total mass of the binder resin and the resin-type dispersant.

### <Optional Components>

This photosensitive composition may include other components to the extent that the effects of the present invention are achieved. The following describes each component that may be included in this photosensitive composition.

### (Thermosetting Compound)

The photosensitive composition may include a thermosetting compound. Thus, when producing a color filter, the heat resistance improves due to increased crosslinking density during the heating process after the film is patterned by photolithography. Additionally, the light-shielding properties further improve because the carbon black becomes less likely to aggregate during the heating process.

Examples of the thermosetting compound include epoxy compounds, oxetane compounds, benzoguanamine compounds, rosin-modified maleic acid compounds, rosin-modified fumaric acid compounds, melamine compounds, urea compounds, and phenol compounds. Among these, epoxy compounds and oxetane compounds are preferable.

### ((Epoxy Compound))

Examples of the epoxy compound include polycondensates of bisphenols (such as bisphenol A, bisphenol F, bisphenol S, biphenol, bisphenol AD, etc.), phenols (such as phenol, alkyl-substituted phenol, aromatic-substituted phenol, naphthol, alkyl-substituted naphthol, dihydroxybenzene, alkyl-substituted dihydroxybenzene, dihydroxynaphthalene, etc.) with various aldehydes (such as formaldehyde, acetaldehyde, alkyl aldehyde, benzaldehyde, alkyl-substituted benzaldehyde, hydroxybenzaldehyde, naphthaldehyde, glutaraldehyde, phthalaldehyde, crotonaldehyde, cinnamaldehyde, etc.); polymers of phenols with various diene compounds (such as dicyclopentadiene, terpenes, vinylcyclohexene, norbornadiene, vinylnorbornene, tetrahydroindene, divinylbenzene, divinylbiphenyl, diisopropenylbiphenyl, butadiene, isoprene, etc.); polycondensates of phenols with ketones (such as acetone, methyl ethyl ketone, methyl isobutyl ketone, acetophenone, benzophenone, etc.); polycondensates of phenols with aromatic dimethanols (such as benzenedimethanol, α,α,α′,α′-benzenedimethanol, biphenyldimethanol, α,α,α′,α′-biphenyldimethanol, etc.); polycondensates of phenols with aromatic dichloromethyls (such as α,α′-dichloroxylene, bischloromethylbiphenyl, etc.); polycondensates of bisphenols with various aldehydes; glycidyl ether-based epoxy resins obtained by glycidylating alcohols; alicyclic epoxy resins; heterocyclic epoxy resins; aliphatic epoxy resins; glycidylamine-based epoxy resins; glycidyl ester-based epoxy resins, and the like.

Examples of commercially available epoxy compounds include Epicoat 807, Epicoat 815, Epicoat 825, Epicoat 827, Epicoat 828, Epicoat 190P, Epicoat 191P (all product names; manufactured by Yuka Shell Epoxy Co., Ltd.), Epicoat 1004, Epicoat 1256 (both product names; manufactured by Japan Epoxy Resin Co., Ltd.), TECHMORE VG3101L (product name; manufactured by Mitsui Chemicals, Inc.), EPPN-501H, 502H (product names; manufactured by Nippon Kayaku Co., Ltd.), JER 1032H60 (product name; manufactured by Japan Epoxy Resin Co., Ltd.), JER 157S65, 157S70 (product names; manufactured by Japan Epoxy Resin Co., Ltd.), EPPN-201 (product name; manufactured by Nippon Kayaku Co., Ltd.), JER152, JER154 (both product names; manufactured by Japan Epoxy Resin Co., Ltd.), EOCN-102S, EOCN-103S, EOCN-104S, EOCN-1020 (all product names; manufactured by Nippon Kayaku Co., Ltd.), Celloxide 2021, EHPE-3150 (both product names; manufactured by Daicel Chemical Industries, Ltd.), Denacol EX-211, 212, 252, 313, 314, 321, 411, 421, 512, 521, 611, 612, 614, 614B, 622, 711, 721 (all product names; manufactured by Nagase ChemteX Corporation), TEPIC-L, TEPIC-H, TEPIC-S (manufactured by Nissan Chemical Industries, Ltd.), and the like.

The content of the epoxy compound is preferably 0.5 parts by mass to 300 parts by mass, and more preferably 1.0 part by mass to 50 parts by mass, with respect to 100 parts by mass of the carbon black. By incorporating an appropriate amount, the heat resistance of the film and the pattern shape are further improved.

### ((Oxetane Compound))

The oxetane compound is a compound having an oxetane group. Examples of the oxetane compound include monofunctional oxetane compounds, bifunctional oxetane compounds, and oxetane compounds with three or more functional groups.

Examples of the monofunctional oxetane compound include (3-ethyloxetan-3-yl)methyl acrylate, (3-ethyloxetan-3-yl)methyl methacrylate, 3-ethyl-3-hydroxymethyloxetane, 3-ethyl-3-(2-ethylhexyloxymethyl)oxetane, 3-ethyl-3-(phenoxymethyl)oxetane, 3-ethyl-3-(2-methacryloyloxymethyl)oxetane, 3-ethyl-3-{[3-(triethoxysilyl)propoxy]methyl}oxetane, and the like.

Commercially available products include OXE-10, OXE-30 manufactured by Osaka Organic Chemical Industry Ltd.), OXT-101, OXT-212 manufactured by Toagosei Co., Ltd.), and the like.

Examples of the bifunctional oxetane compound include 4,4′-bis[(3-ethyl-3-oxetanyl)methoxymethyl]biphenyl, 1,4-bis[(3-ethyl-3-oxetanyl)methoxymethyl]benzene, 1,4-bis{[(3-ethyl-3-oxetanyl)methoxy]methyl}benzene, di[1-ethyl(3-oxetanyl)]methyl ether, di[1-ethyl(3-oxetanyl)]methyl ether-3-ethyl-3-hydroxymethyloxetane, 3-ethyl-3-(2-ethylhexyloxymethyl)oxetane, 3-ethyl-3-(2-phenoxymethyl)oxetane, 3,7-bis(3-oxetanyl)-5-oxa-nonane, 1,2-bis[(3-ethyl-3-oxetanylmethoxy)methyl]ethane, 1,3-bis[(3-ethyl-3-oxetanylmethoxy)methyl]propane, ethylene glycol bis(3-ethyl-3-oxetanylmethyl) ether, dicyclopentenyl bis(3-ethyl-3-oxetanylmethyl) ether, triethylene glycol bis(3-ethyl-3-oxetanylmethyl) ether, tetraethylene glycol bis(3-ethyl-3-oxetanylmethyl) ether, 1,4-bis(3-ethyl-3-oxetanylmethoxy)butane, 1,6-bis(3-ethyl-3-oxetanylmethoxy)hexane, polyethylene glycol bis(3-ethyl-3-oxetanylmethyl) ether, ethylene oxide (EO)-modified bisphenol A bis(3-ethyl-3-oxetanylmethyl) ether, propylene oxide (PO)-modified bisphenol A bis(3-ethyl-3-oxetanylmethyl) ether, EO-modified hydrogenated bisphenol A bis(3-ethyl-3-oxetanylmethyl) ether, PO-modified hydrogenated bisphenol A bis(3-ethyl-3-oxetanylmethyl) ether, EO-modified bisphenol F (3-ethyl-3-oxetanylmethyl) ether, and the like.

Commercially available products include OXBP, OXTP manufactured by Ube Industries, Ltd., OXT-121, OXT-221 manufactured by Toagosei Co., Ltd., and the like.

Examples of the compounds with three or more oxetane functional groups include pentaerythritol tris(3-ethyl-3-oxetanylmethyl) ether, pentaerythritol tetrakis(3-ethyl-3-oxetanylmethyl) ether, dipentaerythritol hexa(3-ethyl-3-oxetanylmethyl) ether, dipentaerythritol pentakis(3-ethyl-3-oxetanylmethyl) ether, dipentaerythritol tetrakis(3-ethyl-3-oxetanylmethyl) ether, caprolactone-modified dipentaerythritol hexa(3-ethyl-3-oxetanylmethyl) ether, caprolactone-modified dipentaerythritol pentakis(3-ethyl-3-oxetanylmethyl) ether, ditrimethylolpropane tetrakis(3-ethyl-3-oxetanylmethyl) ether, resins containing oxetane groups (for example, oxetane-modified phenol novolac resin as described in Japanese Patent No. 3783462), and polymers obtained by radical polymerization of (meth)acrylic monomers such as the aforementioned OXE-30.

The content of the oxetane compound is preferably 0.5 parts by mass to 50 parts by mass, and more preferably 1 part by mass to 40 parts by mass, with respect to 100 parts by mass of carbon black. Including an appropriate amount improves the solvent resistance of the film.

### ((Melamine Compound))

The melamine compound is a compound having a melamine ring structure. The melamine compound includes low molecular weight compounds and high molecular weight compounds. Preferably, the melamine compound is a compound in which a methylol group or an ether group is bonded to the melamine ring. The number of methylol groups and/or ether groups bonded per melamine ring is preferably an average of 5.0 or more. With an appropriate number of bonds, the solvent resistance of the film is further improved, and the contrast ratio becomes less likely to decrease.

Examples of commercially available melamine compounds include Nikalac MW-30HM, MW-390, MW-100LM, MX-750LM, MW-30M, MW-30, MW-22, MS-21, MS-11, MW-24X, MS-001, MX-002, MX-730, MX-750, MX-708, MX-706, MX-042, MX-45, MX-500, MX-520, MX-43, MX-417, MX-410 (manufactured by Sanwa Chemical Co., Ltd.), Cymel 232, 235, 236, 238, 285, 300, 301, 303, 350, 370 (manufactured by Nippon Cytec Industries Inc.), and the like.

Among these, Nikalac MW-30HM, MW-390, MW-100LM, MX-750LM, MW-30M, MW-30, MW-22, MS-21, MS-11, MW-24X, MX-45 (manufactured by Sanwa Chemical Co., Ltd.), and Cymel 232, 235, 236, 238, 300, 301, 303, 350 (manufactured by Nippon Cytec Industries Inc.), which have an average of 5.0 or more methylol groups and/or ether groups per melamine ring, are preferable in terms of further improving the crosslinking density of the film.

In the case of using the thermosetting compound, the total content of the thermosetting compound is preferably 20 parts by mass to 400 parts by mass, and more preferably 50 parts by mass to 250 parts by mass, with respect to 100 parts by mass of carbon black. A content of 20 parts by mass or more is preferable for good film-forming properties and various resistances, and a content of 400 parts by mass or less is preferable because good light-shielding properties can be obtained.

### (Antioxidant)

This photosensitive composition may include an antioxidant. The antioxidant can improve preservation stability, and can prevent the photopolymerization initiator and the thermosetting compound from being oxidized and yellowed by the heat process during heat curing or ITO annealing.

The “antioxidant” may be any compound having an ultraviolet absorption function, a radical scavenging function, or a peroxide decomposition function. Specifically, examples of the antioxidant include hindered phenol-based, hindered amine-based, phosphorus-based, sulfur-based, benzotriazole-based, benzophenone-based, hydroxylamine-based, salicylic acid ester-based, and triazine-based compounds. Known ultraviolet absorbers and antioxidants can be used as the antioxidant.

Among these antioxidants, hindered phenol-based antioxidants, hindered amine-based antioxidants, phosphorus-based antioxidants, or sulfur-based antioxidants are preferable from the viewpoint of balancing both transmittance and sensitivity of the coating film. Hindered phenol-based antioxidants, hindered amine-based antioxidants, or phosphorus-based antioxidants are more preferable.

Examples of the hindered phenol-based antioxidants include 2,4-bis[(laurylthio)methyl]-o-cresol, 1,3,5-tris(3,5-di-t-butyl-4-hydroxybenzyl), 1,3,5-tris(4-t-butyl-3-hydroxy-2,6-dimethylbenzyl), 2,4-bis-(n-octylthio)-6-(4-hydroxy-3,5-di-t-butylanilino)-1,3,5-triazine, pentaerythritol tetrakis[3-(3,5-di-tert-butyl-4-hydroxyphenyl)]propionate, 2,6-di-t-butyl-4-nonylphenol, 2,2′-isobutylidene-bis-(4,6-dimethyl-phenol), 4,4′-butylidene-bis-(2-t-butyl-5-methylphenol), 2,2′-thio-bis-(6-t-butyl-4-methylphenol), 2,5-di-t-amyl-hydroquinone, 2,2′thiodiethylbis-(3,5-di-t-butyl-4-hydroxyphenyl)-propionate, 1,1,3-tris-(2′-methyl-4′-hydroxy-5′-t-butylphenyl)-butane, 2,2′-methylene-bis-(6-(1-methyl-cyclohexyl)-p-cresol), 2,4-dimethyl-6-(1-methyl-cyclohexyl)-phenol, N,N-hexamethylenebis(3,5-di-t-butyl-4-hydroxy-hydrocinnamide), and the like. Other oligomer type and polymer type compounds having hindered phenol structures may also be used.

Examples of the hindered amine-based antioxidants include bis(2,2,6,6-tetramethyl-4-piperidyl) sebacate, bis(N-methyl-2,2,6,6-tetramethyl-4-piperidyl) sebacate, N,N′-bis(2,2,6,6-tetramethyl-4-piperidyl)-1,6-hexamethylenediamine, 2-methyl-2-(2,2,6,6-tetramethyl-4-piperidyl)amino-N-(2,2,6,6-tetramethyl-4-piperidyl)propionamide, tetrakis(2,2,6,6-tetramethyl-4-piperidyl) butane-1,2,3,4-tetracarboxylate, poly[{6-(1,1,3,3-tetramethylbutyl)imino-1,3,5-triazine-2,4-diyl}{(2,2,6,6-tetramethyl-4-piperidyl)imino}hexamethyl{(2,2,6,6-tetramethyl-4-piperidyl)imino}], poly[(6-morpholino-1,3,5-triazine-2,4-diyl){(2,2,6,6-tetramethyl-4-piperidyl)imino}hexamethine{(2,2,6,6-tetramethyl-4-piperidyl)imino}], polycondensates of dimethyl succinate with 1-(2-hydroxyethyl)-4-hydroxy-2,2,6,6-tetramethylpiperidine, N,N′-4,7-tetrakis[4,6-bis{N-butyl-N-(1,2,2,6,6-pentamethyl-4-piperidyl)amino}-1,3,5-triazine-2-yl]-4,7-diazadecane-1,10-diamine, and the like. Other oligomer type and polymer type compounds having hindered amine structures may also be used.

Examples of the phosphorus-based antioxidants include tris(isodecyl) phosphite, tris(tridecyl) phosphite, phenyl isoctyl phosphite, phenyl isodecyl phosphite, phenyl di(tridecyl) phosphite, diphenyl isoctyl phosphite, diphenyl isodecyl phosphite, diphenyl tridecyl phosphite, triphenyl phosphite, tris(nonylphenyl) phosphite, 4,4′-isopropylidenediphenol alkyl phosphite, tris nonylphenyl phosphite, tris dinonylphenyl phosphite, tris(2,4-di-t-butylphenyl) phosphite, tris(biphenyl) phosphite, distearyl pentaerythritol diphosphite, di(2,4-di-t-butylphenyl) pentaerythritol diphosphite, di(nonylphenyl) pentaerythritol diphosphite, phenyl bisphenol A pentaerythritol diphosphite, tetratridecyl 4,4′-butylidenebis(3-methyl-6-t-butylphenol) diphosphite, hexatridecyl 1,1,3-tris(2-methyl-4-hydroxy-5-t-butylphenyl)butane triphosphite, 3,5-di-t-butyl-4-hydroxybenzyl phosphite diethyl ester, sodium bis(4-t-butylphenyl) phosphite, sodium-2,2-methylene-bis(4,6-di-t-butylphenyl)-phosphite, 1,3-bis(diphenoxyphosphonyloxy)-benzene, ethyl phosphite bis(2,4-di-tert-butyl-6-methylphenyl), and the like. Other oligomer type and polymer type compounds having phosphite structures may also be used.

Examples of the sulfur-based antioxidants include 2,2-thio-diethylene bis[3-(3,5-di-t-butyl-4-hydroxyphenyl)propionate], 2,4-bis[(octylthio)methyl]-o-cresol, 2,4-bis[(laurylthio)methyl]-o-cresol, and the like. Other oligomer type and polymer type compounds having thioether structures may also be used.

Examples of the benzotriazole-based antioxidants include oligomer type and polymer type compounds having benzotriazole structures, and the like.

Examples of the benzophenone-based antioxidants include 2-hydroxy-4-methoxybenzophenone, 2,4-dihydroxybenzophenone, 2-hydroxy-4-n-octoxybenzophenone, 4-dodecyloxy-2-hydroxybenzophenone, 2-hydroxy-4-octadecyloxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxy-5-sulfonebenzophenone, 2-hydroxy-4-methoxy-2′-carboxybenzophenone, 2-hydroxy-4-chlorobenzophenone, and the like. Other oligomer type and polymer type compounds having benzophenone structures may also be used.

Examples of the triazine-based antioxidants include 2,4-bis(allyl)-6-(2-hydroxyphenyl)1,3,5-triazine, and the like. Other oligomer type and polymer type compounds having triazine structures may also be used.

Examples of the salicylic acid ester-based antioxidants include phenyl salicylate, p-octylphenyl salicylate, p-tert-butylphenyl salicylate, and the like. Other oligomer type and polymer type compounds having salicylic acid ester structures may also be used.

These antioxidants can be used alone or, as required, in a mixture of two or more types in any ratio.

In addition, the content of the antioxidant is preferably 0.5% by mass to 5.0% by mass in the total non-volatile content of the photosensitive composition, in terms of brightness and sensitivity.

### (Adhesion Promoter)

The photosensitive composition may include an adhesion promoter such as a silane coupling agent to enhance adhesion to the base material. By improving adhesion through the adhesion promoter, the reproducibility of fine lines becomes favorable and the resolution is improved.

Examples of the adhesion promoter include silane coupling agents such as vinyl silanes including vinyltrimethoxysilane and vinyltriethoxysilane; (meth)acryl silanes including 3-methacryloxypropylmethyldimethoxysilane, 3-methacryloxypropyltrimethoxysilane, 3-methacryloxypropylmethyldiethoxysilane, 3-methacryloxypropyltriethoxysilane, and 3-acryloxypropyltrimethoxysilane; epoxy silanes including 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, and 3-glycidoxypropyltriethoxysilane; amino silanes including N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene)propylamine, N-phenyl-3-aminopropyltrimethoxysilane, and hydrochloride of N-(vinylbenzyl)-2-aminoethyl-3-aminopropyltrimethoxysilane; mercaptos including 3-mercaptopropylmethyldimethoxysilane and 3-mercaptopropyltrimethoxysilane; styrils including p-styryltrimethoxysilane; ureidos including 3-ureidopropyltriethoxysilane; sulfides including bis(triethoxysilylpropyl)tetrasulfide; and isocyanates including 3-isocyanatepropyltriethoxysilane, and the like. The adhesion promoter can be used in an amount of 0.01 parts by mass to 10 parts by mass, and preferably 0.05 parts by mass to 5 parts by mass, with respect to 100 parts by mass of the colorant in the colored composition. Within this range, the effects become greater, which is more preferable because the balance of adhesion, resolution, and sensitivity is favorable.

### (Leveling Agent)

The photosensitive composition may include a leveling agent to improve the leveling properties of the composition. It is preferable to use dimethylsiloxane having a polyether structure or polyester structure in the main chain as the leveling agent. Specific examples of the dimethylsiloxane having a polyether structure in the main chain include FZ-2122 manufactured by Toray-Dow Corning, BYK-333 manufactured by BYK Chemie, and the like. Specific examples of the dimethylsiloxane having a polyester structure include BYK-310 and BYK-370 manufactured by BYK Chemie, and the like. Dimethylsiloxane having a polyether structure in the main chain and dimethylsiloxane having a polyester structure in the main chain can also be used in combination. The content ratio of the leveling agent is preferably 0.003% by mass to 0.5% by mass in the total solid content of the colored composition.

As particularly preferable leveling agents, an agent which is a type of surfactant having hydrophobic and hydrophilic groups in the molecule is useful. Such an agent has characteristics of having low solubility in water despite having hydrophilic groups, and exhibiting low surface tension reduction ability when added to the colored composition. Furthermore, despite the low surface tension reduction ability, the agent has good wettability on glass plates. An agent that can sufficiently suppress charging properties at an addition amount that does not cause defects in the coating film due to foaming can be preferably used. Dimethylpolysiloxane having polyalkylene oxide units can be preferably used as the leveling agent having such preferable characteristics. The polyalkylene oxide units include polyethylene oxide units and polypropylene oxide units, and dimethylpolysiloxane may have both polyethylene oxide units and polypropylene oxide units.

The leveling agent may be supplementarily added with anionic, cationic, nonionic, or amphoteric surfactants. Two or more types of surfactants can be used in combination.

Examples of the anionic surfactants that can be supplementarily added to the leveling agent include polyoxyethylene alkyl ether sulfate, sodium dodecylbenzenesulfonate, alkali salt of styrene-acrylic acid copolymer, sodium alkylnaphthalenesulfonate, sodium alkyl diphenyl ether disulfonate, monoethanolamine lauryl sulfate, triethanolamine lauryl sulfate, ammonium lauryl sulfate, monoethanolamine stearate, sodium stearate, sodium lauryl sulfate, monoethanolamine salt of styrene-acrylic acid copolymer, polyoxyethylene alkyl ether phosphate ester, and the like.

Examples of the cationic surfactants that can be supplementarily added to the leveling agent include alkyl quaternary ammonium salt and ethylene oxide adducts thereof. Examples of the nonionic surfactants that can be supplementarily added to the leveling agent include polyoxyethylene oleyl ether, polyoxyethylene lauryl ether, polyoxyethylene nonylphenyl ether, polyoxyethylene alkyl ether phosphate ester, polyoxyethylene sorbitan monostearate, and polyethylene glycol monolaurate; amphoteric surfactants such as alkyl betaines including alkyldimethylaminoacetic acid betaine, and alkyl imidazolines; as well as fluorine-based and silicone-based surfactants.

### (Curing Agent, Curing Accelerator)

The photosensitive composition may include, as required, a curing agent and a curing accelerator to assist in the curing of the thermosetting component. As the curing agent, phenolic resins, amine-based compounds, acid anhydrides, active esters, carboxylic acid compounds, sulfonic acid compounds, and the like are effective, but are not limited to these. Any curing agent that can react with the thermosetting resin may be used. Among these, compounds having two or more phenolic hydroxyl groups in one molecule and amine-based curing agents are preferably. As the aforementioned curing accelerator, for example, amine compounds (for example, dicyandiamide, benzyldimethylamine, 4-(dimethylamino)-N,N-dimethylbenzylamine, 4-methoxy-N,N-dimethylbenzylamine, 4-methyl-N,N-dimethylbenzylamine, etc.), quaternary ammonium salt compounds (for example, triethylbenzylammonium chloride, etc.), blocked isocyanate compounds (for example, dimethylamine, etc.), imidazole derivatives, bicyclic amidine compounds and salts thereof (for example, imidazole, 2-methylimidazole, 2-ethylimidazole, 2-ethyl-4-methylimidazole, 2-phenylimidazole, 4-phenylimidazole, 1-cyanoethyl-2-phenylimidazole, 1-(2-cyanoethyl)-2-ethyl-4-methylimidazole, etc.), phosphorus compounds (for example, triphenylphosphine, etc.), guanamine compounds (for example, melamine, guanamine, acetoguanamine, benzoguanamine, etc.), s-triazine derivatives (for example, 2,4-diamino-6-methacryloyloxy ethyl-s-triazine, 2-vinyl-2,4-diamino-s-triazine, 2-vinyl-4,6-diamino-s-triazine isocyanuric acid adduct, 2,4-diamino-6-methacryloyloxy ethyl-s-triazine isocyanuric acid adduct, etc.), and the like can be used. These may be used alone or in combination of two or more types. The content of the curing accelerator is preferably 0.01 parts by mass to 15 parts by mass, with respect to 100 parts by mass of the thermosetting component.

### (Storage Stabilizer)

The photosensitive composition may include a storage stabilizer to stabilize the viscosity over time.

Examples of the storage stabilizer include quaternary ammonium chlorides such as benzyltrimethyl chloride and diethylhydroxylamine, organic acids and methyl ethers thereof such as lactic acid and oxalic acid, t-butylpyrocatechol, organic phosphines such as tetraethylphosphine and tetraphenylphosphine, phosphites, and the like. The storage stabilizer can be used in an amount of 0.1 parts by mass to 10 parts by mass with respect to 100 parts by mass of the colorant.

### (Solvent)

The photosensitive composition may include a solvent, which facilitates adjustment of the viscosity of the photosensitive composition, making it easy to form a film with a smooth surface. The solvent can be appropriately selected according to the intended use, and included in an appropriate amount.

Examples of the solvent include ester solvents (solvents containing —COO— in the molecule and not containing —O—), ether solvents (solvents containing —O— in the molecule and not containing —COO—), ether ester solvents (solvents containing —COO— and —O— in the molecule), ketone solvents (solvents containing —CO— in the molecule and not containing —COO—), alcohol solvents (solvents containing OH in the molecule and not containing —O—, —CO—, and —COO—), aromatic hydrocarbon solvents, amide solvents, dimethyl sulfoxide, and the like.

Examples of the ester solvents include methyl lactate, ethyl lactate, butyl lactate, methyl 2-hydroxyisobutyrate, ethyl acetate, n-butyl acetate, isobutyl acetate, pentyl formate, isopentyl acetate, butyl propionate, isopropyl butyrate, ethyl butyrate, butyl butyrate, methyl pyruvate, ethyl pyruvate, propyl pyruvate, methyl acetoacetate, ethyl acetoacetate, cyclohexanol acetate, γ-butyrolactone, and the like.

Examples of the ether solvents include ether solvents without hydroxyl groups and ether alcohol solvents (solvents containing —OH and —O— in the molecule). Examples of the ether alcohol solvents include ethylene glycol monomethyl ether, diethylene glycol monomethyl ether, ethylene glycol monoethyl ether, diethylene glycol monoethyl ether, ethylene glycol monopropyl ether, ethylene glycol monobutyl ether, diethylene glycol monomethyl ether, diethylene glycol monoethyl ether, diethylene glycol monobutyl ether, propylene glycol monomethyl ether, dipropylene glycol monomethyl ether, tripropylene glycol monomethyl ether, propylene glycol monoethyl ether, propylene glycol monopropyl ether, propylene glycol monobutyl ether, 3-methoxy-1-butanol, 3-methoxy-3-methylbutanol, and the like.

Examples of the ether solvents without hydroxyl groups include tetrahydrofuran, tetrahydropyran, 1,4-dioxane, diethylene glycol dimethyl ether, diethylene glycol diethyl ether, diethylene glycol methyl ethyl ether, diethylene glycol dipropyl ether, diethylene glycol dibutyl ether, dipropylene glycol dimethyl ether, dipropylene glycol methyl-n-propyl ether, anisole, phenetole, methyl anisole, and the like.

This photosensitive composition preferably includes ether alcohol solvents among these, from the viewpoint of compatibility and coating properties of the photosensitive composition.

Examples of the ether ester solvents include methyl methoxyacetate, ethyl methoxyacetate, butyl methoxyacetate, methyl ethoxyacetate, ethyl ethoxyacetate, methyl 3-methoxypropionate, ethyl 3-methoxypropionate, methyl 3-ethoxypropionate, ethyl 3-ethoxypropionate, methyl 2-methoxypropionate, ethyl 2-methoxypropionate, propyl 2-methoxypropionate, methyl 2-ethoxypropionate, ethyl 2-ethoxypropionate, methyl 2-methoxy-2-methylpropionate, ethyl 2-ethoxy-2-methylpropionate, 3-methoxybutyl acetate, 3-methyl-3-methoxybutyl acetate, propylene glycol monomethyl ether acetate, propylene glycol monoethyl ether acetate, propylene glycol monopropyl ether acetate, ethylene glycol monomethyl ether acetate, ethylene glycol monoethyl ether acetate, diethylene glycol monoethyl ether acetate, diethylene glycol monobutyl ether acetate, dipropylene glycol methyl ether acetate, dipropylene glycol diacetate, and the like.

Examples of the ketone solvents include 4-hydroxy-4-methyl-2-pentanone, acetone, 2-butanone, 2-heptanone, 3-heptanone, 4-heptanone, 4-methyl-2-pentanone, cyclopentanone, cyclohexanone, isophorone, and the like.

Examples of the alcohol solvents include methanol, ethanol, propanol, butanol, hexanol, cyclohexanol, ethylene glycol, propylene glycol, 1,3-butylene glycol, glycerin, and the like.

Examples of the aromatic hydrocarbon solvents include benzene, toluene, xylene, mesitylene, and the like.

Examples of the amide solvents include N,N-dimethylformamide, N,N-dimethylacetamide, N-methylpyrrolidone, and the like.

Among these, solvents with a boiling point of 120° C. or higher and 245° C. or lower at 1 atm are preferable in terms of coating properties and drying properties. For example, propylene glycol monomethyl ether acetate, ethyl lactate, butyl lactate, propylene glycol monomethyl ether, ethyl 3-ethoxypropionate, ethylene glycol monomethyl ether, diethylene glycol monomethyl ether, diethylene glycol monoethyl ether, 4-hydroxy-4-methyl-2-pentanone, N,N-dimethylformamide, N-methylpyrrolidone, cyclohexanone, tripropylene glycol monomethyl ether, 3-methoxy-1-butanol, 3-methoxy-3-methylbutanol, diethylene glycol monomethyl ether, diethylene glycol monoethyl ether, diethylene glycol monobutyl ether, propylene glycol monomethyl ether, propylene glycol monobutyl ether, dipropylene glycol monomethyl ether, and the like are more preferable, and propylene glycol monomethyl ether acetate, propylene glycol monomethyl ether, ethyl lactate, ethyl 3-ethoxypropionate, cyclohexanone, tripropylene glycol monomethyl ether, 3-methoxy-1-butanol, and the like are even more preferable.

Moreover, these solvents are preferably used in an amount of 200 parts by mass to 900 parts by mass, and more preferably 300 parts by mass to 570 parts by mass, with respect to 100 parts by mass of the non-volatile content in the photosensitive composition, as these solvents can adjust the photosensitive composition to an appropriate viscosity and form a coating film with a uniform film thickness as intended. The viscosity of the photosensitive composition is preferably 2.4 mPa·s to 7.2 mPa·s, and more preferably 3.4 mPa·s to 6.4 mPa·s.

### (Dispersion Aid)

When dispersing carbon black, etc., a dispersion aid such as a pigment derivative and a surfactant may be appropriately used in place of or in combination with the aforementioned resin-type dispersant. Since the dispersion aid has significant effects in preventing re-agglomeration of carbon black after dispersion, the photosensitive composition using the dispersion aid exhibits good viscosity stability, and the resulting cured film has excellent light-shielding properties.

### ((Pigment Derivative))

Examples of the pigment derivative include compounds in which basic substituents, acidic substituents, or phthalimide methyl groups that may have substituents are introduced to organic pigments, anthraquinone, acridone, or triazine. For example, compounds described in Japanese Patent Application Laid-Open No. 563-305173, Japanese Patent Publication No. 557-15620, Japanese Patent Publication No. S59-40172, Japanese Patent Publication No. S63-17102, Japanese Patent Publication No. H5-9469, Japanese Patent Application Laid-Open No. 2001-335717, Japanese Patent Application Laid-Open No. 2003-128669, Japanese Patent Application Laid-Open No. 2004-091497, Japanese Patent Application Laid-Open No. 2007-156395, Japanese Patent Application Laid-Open No. 2008-094873, Japanese Patent Application Laid-Open No. 2008-094986, Japanese Patent Application Laid-Open No. 2008-095007, Japanese Patent Application Laid-Open No. 2008-195916, and Japanese Patent No. 4585781 can be used, and these can be used alone or in a mixture of two or more types.

The content of the pigment derivative is preferably 0.5 parts by mass or more, more preferably 1 part by mass or more, and most preferably 3 parts by mass or more, with respect to 100 parts by mass of carbon black, from the viewpoint of improving dispersibility. In addition, from the viewpoint of heat resistance and light resistance, the content is preferably 40 parts by mass or less, and more preferably 35 parts by mass or less.

### ((Surfactant))

Examples of the surfactant include anionic surfactants such as sodium lauryl sulfate, polyoxyethylene alkyl ether sulfate, sodium dodecylbenzenesulfonate, alkali salts of styrene-acrylic acid copolymers, sodium stearate, sodium alkylnaphthalenesulfonate, sodium alkyl diphenyl ether disulfonate, monoethanolamine lauryl sulfate, triethanolamine lauryl sulfate, ammonium lauryl sulfate, monoethanolamine stearate, monoethanolamine salts of styrene-acrylic acid copolymers, and polyoxyethylene alkyl ether phosphate; nonionic surfactants such as polyoxyethylene oleyl ether, polyoxyethylene lauryl ether, polyoxyethylene nonylphenyl ether, polyoxyethylene alkyl ether phosphate, polyoxyethylene sorbitan monostearate, and polyethylene glycol monolaurate; cationic surfactants such as alkyl quaternary ammonium salt and ethylene oxide adducts thereof; and amphoteric surfactants such as alkyl betaines including alkyldimethylaminoacetic acid betaine, alkyl imidazolines, and the like. These can be used alone or in a mixture of two or more types, but are not necessarily limited to these.

In the case of using a resin-type dispersant and a surfactant, the content ratio thereof is preferably 0.1 parts by mass to 55 parts by mass, and more preferably 0.1 parts by mass to 45 parts by mass, with respect to 100 parts by mass of carbon black. In the case where the content is less than 0.1 parts by mass, it is difficult to obtain the effects of addition, and if the content exceeds 55 parts by mass, the excess dispersant may affect the dispersion.

### (Thiol-Based Chain Transfer Agent)

The photosensitive composition may include a thiol-based chain transfer agent as a chain transfer agent. By using a thiol together with the photopolymerization initiator, the thiol acts as a chain transfer agent in the radical polymerization process after light irradiation, which generates thiyl radicals that are less susceptible to polymerization inhibition by oxygen, thereby improving the sensitivity of the photosensitive composition.

The thiol-based chain transfer agent is preferably a multifunctional thiol having two or more thiol groups (SH groups), and more preferably having three or more SH groups. As the number of functional groups increases, it becomes easier for the film to be photocured from the surface to the deepest part.

Examples of the multifunctional thiol include hexanedithiol, decanedithiol, 1,4-butanediol bisthiopropionate, 1,4-butanediol bisthioglycolate, ethylene glycol bisthioglycolate, ethylene glycol bisthiopropionate, trimethylolpropane tristhioglycolate, trimethylolpropane tristhiopropionate, trimethylolpropane tris(3-mercaptobutyrate), pentaerythritol tetrakisthioglycolate, pentaerythritol tetrakisthiopropionate, trimercaptopropionic acid tris(2-hydroxyethyl)isocyanurate, 1,4-dimethylmercaptobenzene, 2,4,6-trimercapto-s-triazine, 2-(N,N-dibutylamino)-4,6-dimercapto-s-triazine, and the like. Ethylene glycol bisthiopropionate, trimethylolpropane tristhiopropionate, and pentaerythritol tetrakisthiopropionate are preferable.

The thiol-based chain transfer agent can be used alone or in combination of two or more types.

The content of the thiol-based chain transfer agent is preferably 0.5% to 10% in the total non-volatile content of the photosensitive composition, and more preferably 1% to 8%. Including an appropriate amount improves taper shape, wrinkles, film shrinkage rate, light sensitivity, and pattern shape.

### <Manufacturing Method of Photosensitive Composition>

The manufacturing method of the photosensitive composition includes, for example, a method of first preparing a carbon black dispersion, and then adding a polymerizable compound, a photopolymerization initiator, a photocurable resin, and other components used as required.

The carbon black dispersion can be prepared by finely dispersing the colorant in a colorant carrier such as a binder resin and/or an organic solvent, preferably together with a dispersion aid, using various dispersion means such as a kneader, a two-roll mill, a three-roll mill, a ball mill, a horizontal sand mill, a vertical sand mill, an annular bead mill, or an attritor. In the case of using a colorant different from carbon black, the colorant and carbon black may be dispersed simultaneously, or dispersions may be prepared separately and then mixed. Furthermore, in the case where the solubility of the colorant is high, the colorant may be dissolved by stirring, and the above-mentioned dispersion process may not be performed.

Additionally, it is preferable to remove coarse particles of 5 μm or larger, preferably 1 μm or larger, more preferably 0.5 μm or larger, and particularly preferably 0.3 μm or larger, and mixed dust from the obtained composition by means such as centrifugation, filtration using a sintered filter or a membrane filter, and the like.

### 2. Organic EL Display Device

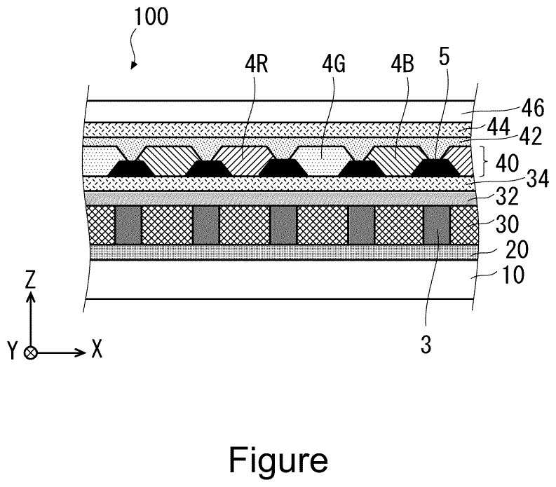

Referring to FIG. 1, the structure of the organic EL display device of the present disclosure (hereinafter also referred to as this organic EL display device) will be described. The organic EL display device 100 shown in the example of FIG. 1 includes a substrate 10, a pixel electrode 20, an organic EL layer 30, and a color filter layer 40. The color filter layer 40 has red pixels 4R, green pixels 4G, and blue pixels 4B in a pattern, and a black matrix 5 is further arranged between the color pixels. Further, in the example of FIG. 1, a sealing layer 32 and an adhesive layer 34 are provided between the organic EL layer 30 and the color filter layer 40, and an overcoat layer 42, an adhesive layer 44, and a protective layer 46 are provided on the display surface side of the color filter layer 40. In addition, a partition wall 3 corresponding to each pixel is arranged in the organic EL layer 30. Although not shown in the FIGURE, a wiring pattern is formed on the substrate 10 in the outer peripheral portion of the display area. A light-shielding portion may be provided on the wiring pattern.

This organic EL display device excels in light-shielding properties because the light-shielding layer including the aforementioned black matrix 5 and the aforementioned light-shielding portion is a cured film formed from this photosensitive composition, and can also suppress reflected light originating from the wiring pattern.

The substrate 10 is a substrate on which the pixel electrode 20 is formed, and a silicon substrate is preferable. The pixel electrode 20 is an electrode for driving the organic EL layer, and is, for example, a TFT (Thin Film Transistor) array. The protective layer 46 includes cover glass and known hard coat layers.

The organic EL layer 30 is a single layer or multiple layers of an organic light-emitting material that contains a light-emitting substance. In the case of multiple layers, for example, the organic EL layer 30 may have a three-layer structure composed of a hole transport layer, an electron-transporting organic light-emitting layer, and an electron transport layer sequentially stacked, may include separate layers for injection function and transport function such as a hole (electron) injection layer and a hole (electron) transport layer, or may include a layer that blocks hole (electron) transport.

An example of the organic EL layer includes a structure in which an anode, an organic layer, and a cathode are sequentially stacked from the silicon substrate side, and are hermetically covered with a sealing layer.

The anode is provided on the silicon substrate and is composed of a conductive material with a large work function. Examples of the conductive material with a large work function include nickel, silver, gold, platinum, palladium, selenium, rhodium, ruthenium, iridium, rhenium, tungsten, molybdenum, chromium, tantalum, niobium, and alloys thereof, or tin oxide (SnO₂), indium tin oxide (ITO), zinc oxide, titanium oxide, and the like.

The cathode is composed of a conductive material with a small work function. Examples of such a conductive material include alloys of active metals such as Li, Mg, and Ca with metals such as Ag, Al, and In, or structures in which these are stacked. Additionally, a structure may be used in which a thin compound layer of active metals such as Li, Mg, and Ca with halogens such as fluorine and bromine, or oxygen is inserted between the organic layer and the cathode.

The anode and the cathode are patterned into suitable shapes according to the driving method of the display device. For example, in the case where the driving method of the organic EL display device is a simple matrix type, the anode and the cathode are formed in intersecting stripe shapes, and the intersecting portions become the organic EL elements.

The organic layer has at least a white light-emitting layer, but is typically composed of multiple organic layers. The organic layer can have charge injection layers such as a hole injection layer and an electron injection layer, as well as charge transport layers such as a hole transport layer that transports holes to the white light-emitting layer and an electron transport layer that transports electrons to the white light-emitting layer. The organic layer can have any known configuration that includes a white light-emitting layer.

The light-emitting layer can use any known material that produces white light emission. The white light emission characteristics may have emission in at least three regions, a red region (600 nm to 780 nm), a green region (475 nm to 600 nm), and a blue region (380 nm to 475 nm). The emission peaks are not necessarily three or more in number. For example, even with two emission peaks, it is sufficient if there is emission in the aforementioned regions. However, to obtain a wide color reproducibility, it is preferable to use a white light-emitting layer with three or more emission peaks, and it is preferable to have emission peaks in one or more of the aforementioned three color regions.

The materials constituting such a white light-emitting layer are not particularly limited as long as the materials emit fluorescence or phosphorescence. Additionally, the light-emitting materials may have hole transport properties or electron transport properties. Examples of the light-emitting materials include dye-based materials, metal complex-based materials, and polymer-based materials.

Examples of the dye-based materials include cyclopentadiene derivatives, tetraphenylbutadiene derivatives, triphenylamine derivatives, oxadiazole derivatives, pyrazoloquinoline derivatives, distyrylbenzene derivatives, distyryl arylene derivatives, silole derivatives, thiophene ring compounds, pyridine ring compounds, perinone derivatives, perylene derivatives, oligothiophene derivatives, triphenylamine derivatives, oxadiazole dimers, and pyrazoline dimers.

Examples of the metal complex-based materials include aluminum quinolinol complexes, benzoquinolinol beryllium complexes, benzoxazole zinc complexes, benzothiazole zinc complexes, azomethine zinc complexes, porphyrin zinc complexes, and europium complexes, or metal complexes having Al, Zn, Be, etc., or rare earth metals such as Tb, Eu, and Dy as the central metal, and having ligands with structures such as oxadiazole, thiadiazole, phenylpyridine, phenylbenzoimidazole, and quinoline.

Examples of the polymer-based materials include polyphenylene vinylene derivatives, polythiophene derivatives, polyparaphenylene derivatives, polysilane derivatives, polyacetylene derivatives, polyfluorene derivatives, polyvinylcarbazole derivatives, as well as polymerized forms of the aforementioned dye-based materials and metal complex-based materials.

Examples of the methods for forming the aforementioned white light-emitting layer include vapor deposition, printing, inkjet, or spin coating, casting, dipping, bar coating, blade coating, roll coating, gravure coating, flexographic printing, spray coating, self-assembly methods (alternating adsorption method, self-assembled monolayer method), etc. It is particularly preferable to use vapor deposition, spin coating, and inkjet. The film thickness of the white light-emitting layer is typically in the range of about 5 nm to 5 μm.

The organic EL layer may also include a hole injection layer between the white light-emitting layer and the anode. By providing the hole injection layer, the injection of holes into the white light-emitting layer can be stabilized, thereby increasing the light emission efficiency. The material for forming the hole injection layer can be materials generally used for hole injection layers in organic EL elements. Additionally, the material for forming the hole injection layer may have either hole injection properties or electron barrier properties.

Specifically, examples of the material for forming the hole injection layer include triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, polysilane-based or aniline-based copolymers, conductive polymer oligomers such as thiophene oligomers, and the like.

Furthermore, examples of the material for forming the hole injection layer include porphyrin compounds, aromatic tertiary amine compounds, and styrylamine compounds. The film thickness of the hole injection layer is typically in the range of about 5 nm to 1 μm.

The organic EL layer may also include an electron injection layer formed between the white light-emitting layer and the cathode. By providing the electron injection layer, the injection of electrons into the white light-emitting layer can be stabilized, thereby increasing the light emission efficiency.

Examples of the material for forming the electron injection layer include nitro-substituted fluorene derivatives, anthraquinodimethane derivatives, diphenylquinone derivatives, thiopyran dioxide derivatives, heterocyclic tetracarboxylic acid anhydrides such as naphthaleneperylene, carbodiimides, fluorenylidene methane derivatives, anthraquinodimethane and anthrone derivatives, oxadiazole derivatives, thiazole derivatives in which the oxygen atom of the oxadiazole ring of oxadiazole derivatives is substituted with a sulfur atom, quinoxaline derivatives having a quinoxaline ring known as an electron-withdrawing group, metal complexes of 8-quinolinol derivatives such as tris(8-quinolinolato)aluminum, phthalocyanine, metal phthalocyanines, distyrylpyrazine derivatives, and the like.

The color filter layer 40 is a layer arranged directly on the organic EL layer 30, or via other layers such as the adhesive layer 34, and includes red pixels 4R, green pixels 4G, blue pixels 4B, and the black matrix 5 between the color pixels. The color filter layer may also have magenta pixels, cyan pixels, yellow pixels, or other pixels.

In terms of high resolution, the area per pixel is preferably 1 μm² to 50 μm², and more preferably 1 μm² to 30 μm². In addition, the thickness of each color pixel is preferably 0.1 μm to 5 μm, and more preferably 0.2 μm to 4 μm, in terms of color reproducibility.

### <Manufacturing Method of Organic EL Display Device>

In manufacturing the organic EL display device, first, a laminate including the substrate 10, the pixel electrode 20, and the organic EL layer 30 is prepared. The manufacturing method for each layer may be appropriately selected from known methods. The color filter layer 40 may be formed directly on the organic EL layer 30, or the sealing layer 32 and/or the adhesive layer 34 may be formed on the organic EL layer 30, and the color filter layer 40 is then formed on the sealing layer 32 or the adhesive layer 34. For the sealing layer 32, known curable resins can be used, with UV-curable resins being preferable, and thermosetting resins may also be used in combination. Although not particularly limited, acrylic resins sensitive to i-line (wavelength 365 nm) are preferable as the UV-curable resins. Forming the sealing layer can planarize the surface on which the color filter layer is formed.

The photolithography method is preferable for forming the color filter layer 40. In the case of the photolithography method, in addition to the aforementioned photosensitive composition, photosensitive compositions for red, green, blue, etc. are prepared.

The color filter layer 40 typically begins with forming a light-shielding layer including the black matrix 5. The black matrix 5 is formed by applying the photosensitive composition on the organic EL layer 30, the sealing layer 32, or the adhesive layer 34 to form a coating film, exposing, developing, and heat-curing.

The above applying method can be appropriately selected from known methods such as spray coating, dip coating, bar coating, roll coating, and spin coating.

The above exposing method includes, for example, a method of using ultraviolet rays from low-pressure mercury lamps, high-pressure mercury lamps, metal halide lamps, or electron beams as the light source, and exposing through a mask with a predetermined pattern. A patterned light-shielding layer can be obtained by removing the uncured photosensitive composition using a developer such as an alkaline developer.

From the viewpoint of protecting the organic EL layer, the temperature of the above heat-curing is preferably 120° C. or lower, and more preferably 100° C. or lower. By using the photosensitive composition, sufficient curing can be achieved even at a low temperature of 120° C. or lower.

The film thickness of the black matrix (light-shielding layer) is preferably 0.1 μm to 5 μm, more preferably 0.5 μm to 2.0 μm, and even more preferably 1.0 μm to 2.0 μm. The film thickness of the black matrix and the film thickness of the light-shielding portion provided in the outer peripheral portion of the element may be the same or different.

Next, pixels of each color are formed by using red, green, and blue photosensitive compositions to form coating films, exposing, developing, and heat-curing. Further, the overcoat layer 42, the adhesive layer 44, the protective layer 46, etc. may be formed on the display surface side of the color filter layer 40 by known methods.

As a result, an organic EL display device including a light-shielding layer with excellent light-shielding properties can be obtained.

### EXAMPLES

The present invention will be described below based on examples. In the examples, “parts” and “%” represent “parts by weight” and “% by weight”, respectively. The present invention is not limited to the examples.

### <Manufacture of Resin-Type Dispersant Solution>

### (Resin-Type Dispersant Solution 1): Photosensitive Resin

A reaction vessel equipped with a gas introduction tube, a thermometer, a condenser, and a stirrer was charged with 8 parts of 3-mercapto-1,2-propanediol, 12 parts of pyromellitic anhydride, 80 parts of propylene glycol monomethyl ether acetate (PGMAc), and 0.2 parts of monobutyltin oxide as a catalyst. After replacing the atmosphere with nitrogen gas, the mixture was reacted at 120° C. for 5 hours (first step). It was confirmed by acid value measurement that 95% or more of the acid anhydride had undergone half-esterification. Subsequently, 15 parts of methyl methacrylate (MMA), 10 parts of t-butyl acrylate (tBA), 10 parts of ethyl acrylate (EA), 5 parts of methacrylic acid (MAA), 10 parts of benzyl methacrylate (BzMA), and 50 parts of 2-hydroxyethyl methacrylate (HEMA) were added. The inside of the reaction vessel was heated to 80° C., 1 part of 2,2′-azobis(2,4-dimethylvaleronitrile) was added, and the mixture was reacted for 12 hours (second step). It was confirmed by solid content measurement that 95% had reacted. Next, the atmosphere in the flask was replaced with air, 54.0 parts of 2-methacryloyloxyethyl isocyanate (MOI) and 0.1 parts of hydroquinone were added, and the mixture was reacted at 70° C. for 4 hours (third step). After confirming the disappearance of the peak at 2270 cm⁻¹ based on the isocyanate group by IR, the reaction solution was cooled and adjusted for solid content with PGMAc to obtain the resin-type dispersant solution 1, which was a photosensitive resin with 40% solid content. The acid value of the obtained resin-type dispersant was 36, and the weight average molecular weight was 12,000.

### (Resin-Type Dispersant Solution 2): Non-Photosensitive

A reaction vessel equipped with a gas introduction tube, a thermometer, a condenser, and a stirrer was charged with 30 parts of ethyl acrylate, 20 parts of tert-butyl acrylate, and 40 parts of 2-methyl methacrylate, and the atmosphere was replaced with nitrogen gas. The inside of the reaction vessel was heated to 80° C., and a solution of 0.1 parts of 2,2′-azobisisobutyronitrile dissolved in 45.7 parts of cyclohexanone was added to 6 parts of 3-mercapto-1,2-propanediol, and reacted for 10 hours. It was confirmed by solid content measurement that 95% had reacted. At this point, the weight average molecular weight was 4000. Subsequently, 9.7 parts of pyromellitic dianhydride, 70 parts of PGMAc, and 0.20 parts of 1,8-diazabicyclo-[5.4.0]-7-undecene as a catalyst were added, and the mixture was reacted at 120° C. for 7 hours. It was confirmed by acid value measurement that 98% or more of the acid anhydride had undergone half-esterification, and the reaction was completed. After the reaction was completed, the non-volatile content was adjusted to 40% by weight, and a PGMAc solution of resin-type dispersant (resin-type dispersant solution 2) with a weight average molecular weight of 8,100, an acid value of 50 mgKOH/g, and a glass transition temperature of 22.5° C. for the vinyl polymerization portion was obtained.

### <Manufacture of Binder Resin>

### (Acrylic Resin Solution (D-1)): Photosensitive Resin

A reaction vessel with a thermometer, a cooling tube, a nitrogen gas introduction tube, and a stirring device attached to a separable 4-neck flask was charged with 100 parts of propylene glycol monomethyl ether acetate, and while introducing nitrogen gas into the vessel, the reaction vessel was heated to 120° C. At the same temperature, a mixture of 5.2 parts of styrene, 35.5 parts of glycidyl methacrylate, 41.0 parts of dicyclopentanyl methacrylate, and 1.0 part of azobisisobutyronitrile was added dropwise over 2.5 hours through a dropper to perform the polymerization reaction. Next, the atmosphere in the flask was replaced with air, and 17.0 parts of acrylic acid, 0.3 parts of tris(dimethylaminomethyl)phenol, and 0.3 parts of hydroquinone were added. The reaction was continued at 120° C. for 5 hours, and when the solid content acid value reached 0.8, the reaction was completed. A resin solution with a weight average molecular weight of approximately 12,000 (measured by GPC (gel permeation chromatography)) was obtained.

Furthermore, 30.4 parts of tetrahydrophthalic anhydride and 0.5 parts of triethylamine were added, and the mixture was reacted at 120° C. for 4 hours. Propylene glycol monomethyl ether acetate was added to adjust the non-volatile content to 20%, thereby preparing the acrylic resin solution (D-1).

### (Acrylic Resin Solution (D-2)): Photosensitive Resin

A flask equipped with a stirrer, a thermometer, a reflux condenser, a dropping funnel, and a nitrogen introduction tube was charged with 182 g of propylene glycol monomethyl ether acetate. After changing the atmosphere in the flask from air to nitrogen, the temperature was raised to 100° C. Then, a solution of a mixture of 70.5 g (0.40 mol) of benzyl methacrylate, 43.0 g (0.5 mol) of methacrylic acid, 22.0 g (0.10 mol) of monomethacrylate with a tricyclodecane skeleton (FA-513M manufactured by Hitachi Chemical Co., Ltd.), and 136 g of propylene glycol monomethyl ether acetate, to which 3.6 g of azobisisobutyronitrile was added, was added dropwise to the flask from the dropping funnel over 2 hours. The mixture was further stirred at 100° C. for 5 hours. Next, the atmosphere in the flask was changed from nitrogen to air, and 35.5 g [0.25 mol, (50 mol % relative to the carboxyl groups of methacrylic acid used in this reaction)] of glycidyl methacrylate, 0.9 g of tris(dimethylaminomethyl)phenol, and 0.145 g of hydroquinone were added to the flask. The reaction was continued at 110° C. for 6 hours, and propylene glycol monomethyl ether acetate was added to adjust the non-volatile content to 20%, thereby obtaining an acrylic resin solution (D-2) with a solid content acid value of 79 mgKOH/g. The weight average molecular weight measured by GPC in polystyrene equivalent was 13,000, and the molecular weight distribution (Mw/Mn) was 2.1.

### (Acrylic Resin Solution (D-3)): Non-Photosensitive

A reaction vessel with a thermometer, a condenser, a nitrogen gas introduction tube, a dropper, and a stirring device attached to a separable 4-neck flask was charged with 196 parts of cyclohexanone, and the temperature was raised to 80° C. After replacing the atmosphere in the reaction vessel with nitrogen, a mixture of 20.0 parts of benzyl methacrylate, 17.2 parts of n-butyl methacrylate, 12.9 parts of 2-hydroxyethyl methacrylate, 12.0 parts of methacrylic acid, 20.7 parts of paracumylphenol ethylene oxide modified acrylate (ARONIX M110 manufactured by Toagosei Co., Ltd.), and 1.1 parts of 2,2′-azobisisobutyronitrile was added dropwise through the dropper over 2 hours. After the addition was completed, the reaction was further continued for 3 hours to obtain a solution of acrylic resin.

After cooling to room temperature, approximately 2 parts of the resin solution were sampled, and heated and dried at 180° C. for 20 minutes to measure the non-volatile content. Propylene glycol monomethyl ether acetate was added to the previously synthesized resin solution to adjust the non-volatile content to 20% by mass, thereby preparing the acrylic resin solution (D-3). The weight average molecular weight (Mw) was 26,000.

### <Manufacture of Carbon Black Dispersion>

### (Dispersion (PK-1))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-1) with 20% by weight of non-volatile content.
Carbon black (manufactured by Mitsubishi Chemical_#40):15.0 parts
Resin-type dispersant solution 1 (photosensitive resin):12.5 parts
Propylene glycol monomethyl ether acetate:70.0 parts

### (Dispersion (PK-2))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-2) with 20% by weight of non-volatile content.
Carbon black (manufactured by Mitsubishi Chemical_#45):15.0 parts
Resin-type dispersant solution 1 (photosensitive resin):12.5 parts
Propylene glycol monomethyl ether acetate:70.0 parts

### (Dispersion (PK-3))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-3) with 20% by weight of non-volatile content.
Carbon black (manufactured by Mitsubishi Chemical_#45L):15.0 parts
Resin-type dispersant solution 1 (photosensitive resin):12.5 parts
Propylene glycol monomethyl ether acetate:70.0 parts

### (Dispersion (PK-4))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-4) with 20% by weight of non-volatile content.
Carbon black (manufactured by Orion Engineered Carbons_Printex55):15.0 parts
Resin-type dispersant solution 1 (photosensitive resin):12.5 parts
Propylene glycol monomethyl ether acetate:70.0 parts

### (Dispersion (PK-5))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-5) with 20% by weight of non-volatile content.
Carbon black (manufactured by Mitsubishi Chemical_#47):15.0 parts
Resin-type dispersant solution 1 (photosensitive resin):12.5 parts
Propylene glycol monomethyl ether acetate:70.0 parts

### (Dispersion (PK-6))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-6) with 20% by weight of non-volatile content.
Carbon black (manufactured by Mitsubishi Chemical_#52):15.0 parts
Resin-type dispersant solution 1 (photosensitive resin):12.5 parts
Propylene glycol monomethyl ether acetate:70.0 parts

### (Dispersion (PK-7))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-7) with 20% by weight of non-volatile content.
Carbon black (manufactured by Mitsubishi Chemical_#44):15.0 parts
Resin-type dispersant solution 1 (photosensitive resin):12.5 parts
Propylene glycol monomethyl ether acetate:70.0 parts

### (Dispersion (PK-8))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-8) with 20% by weight of non-volatile content.
Carbon black (manufactured by Mitsubishi Chemical_#33):15.0 parts
Resin-type dispersant solution 1 (photosensitive resin):12.5 parts
Propylene glycol monomethyl ether acetate:70.0 parts

### (Dispersion (PK-9))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-9) with 20% by weight of non-volatile content.
Carbon black (manufactured by Mitsubishi Chemical_#5):15.0 parts
Resin-type dispersant solution 1 (photosensitive resin):12.5 parts
Propylene glycol monomethyl ether acetate:70.0 parts

### (Dispersion (PK-10))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-10) with 20% by weight of non-volatile content.
Carbon black (manufactured by Mitsubishi Chemical_#1000):15.0 parts
Resin-type dispersant solution 1 (photosensitive resin):12.5 parts
Propylene glycol monomethyl ether acetate:70.0 parts

### (Dispersion (PK-11))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-11) with 20% by weight of non-volatile content.
Carbon black (manufactured by Mitsubishi Chemical_#40):15.0 parts
Resin-type dispersant solution 1 (photosensitive resin):12.5 parts
Propylene glycol monomethyl ether acetate:70.0 parts

### (Dispersion (PK-12))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-12) with 20% by weight of non-volatile content.
Carbon black (manufactured by Mitsubishi Chemical_#40):15.0 parts
Resin-type dispersant solution 1 (photosensitive resin):6.25 parts
Resin-type dispersant solution 2 (non-photosensitive resin):6.25 parts
Propylene glycol monomethyl ether acetate:70.0 parts

### (Dispersion (PK-13))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-13) with 20% by weight of non-volatile content.
Carbon black (manufactured by Mitsubishi Chemical_#40):15.0 parts
Resin-type dispersant solution 1 (photosensitive resin):9.4 parts
Resin-type dispersant solution 2 (non-photosensitive resin):3.1 parts
Propylene glycol monomethyl ether acetate:70.0 parts

### (Dispersion (PK-14))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-14) with 20% by weight of non-volatile content.

C.I. Pigment Yellow 139 (“Paliotol Yellow D1819” manufactured by BASF Japan):3.75 parts
C.I. Pigment Red 254 (“Irgajin RedL 3630” manufactured by BASF Japan):2.85 parts
C.I. Pigment Violet 23 (“LIONOGEN VIOLET RL” manufactured by Toyo Color):0.90 parts
C.I. Pigment Blue 15:6 (“LIONOL BLUE ES” manufactured by Toyo Color):7.50 parts
Resin-type dispersant solution 1 (photosensitive resin):12.5 parts
Propylene glycol monomethyl ether acetate:70.0 parts

### (Dispersion (PK-15))

After stirring and mixing the following mixture to be uniform, the mixture was dispersed for 3 hours using an eigermill (“Mini Model M-250 MKII” manufactured by Eiger Japan) with zirconia beads of 0.5 mm in diameter. The mixture was then filtered through a 5.0 μm filter to prepare a pigment dispersion (PK-15) with 20% by weight of non-volatile content.
Perylene-based black pigment “Lumogen Black FK4280” manufactured by BASF Japan:15.0 parts
Resin-type dispersant solution 1 (photosensitive resin):12.5 parts
Propylene glycol monomethyl ether acetate:70.0 parts

### <Manufacture of Photosensitive Composition>

### Example 1

### (Black Photosensitive Composition (R-4))

After stirring and mixing the following mixture to be uniform, the mixture was filtered through a 0.5 μm filter to obtain the photosensitive composition (R-4).
Dispersion (PK-1):56.0 parts
Polymerizable compound (B-1):3.0 parts
Polymerizable compound (B-2):3.0 parts
Photopolymerization initiator (C-1):2.8 parts
Leveling agent (I % non-volatile content):1.00 part
Solvent (S-1):34.2 parts

### Examples 2 to 28, Comparative Examples 1 to 6

### (Preparation of Photosensitive Compositions (R-1 to R-3, R-5 to R-34))

Photosensitive compositions were obtained in the same manner as the photosensitive composition (R-4), except that the types and amounts of the pigment dispersion, polymerizable compound, photopolymerization initiator, and resin solution were changed to the contents shown in Table 1 to Table 4.

TABLE 1
Black photosensitive colored composition

| | Comparative | Comparative | Comparative | Example | Example | Example | Example | Example | Example | Example |
|---|---|---|---|---|---|---|---|---|---|---|
| | Example 1 | Example 2 | Example 3 | 1 | 2 | 3 | 4 | 5 | 6 | 7 |
| | R-1 | R-2 | R-3 | R-4 | R-5 | R-6 | R-7 | R-8 | R-9 | R-10 |
| Pigment dispersion (A) | PK-1 | PK-1 | PK-1 | PK-1 | PK-1 | PK-1 | PK-1 | PK-1 | PK-1 | PK-1 |
| | 56.0 | 56.0 | 56.0 | 56.0 | 56.0 | 56.0 | 56.0 | 56.0 | 56.0 | 56.0 |
| Photopolymerizable | B-1 | B-2 | B-3 | B-1 | B-1 | B-1 | B-1 | B-1 | B-1 | B-1 |
| monomer 1 | 6.0 | 6.0 | 6.0 | 3.0 | 3.0 | 3.0 | 3.0 | 3.0 | 3.0 | 3.0 |
| Photopolymerizable | | | | B-2 | B-3 | B-2 | B-2 | B-2 | B-2 | B-2 |
| monomer 2 | | | | 3.0 | 3.0 | 3.0 | 3.0 | 3.0 | 3.0 | 3.0 |
| Photopolymerization | C-1 | C-1 | C-1 | C-1 | C-1 | C-1 | C-1 | C-1 | C-1 | C-1 |
| initiator (C) | 2.8 | 2.8 | 2.8 | 2.8 | 2.8 | 2.8 | 2.8 | 2.8 | 2.8 | 2.8 |
| Resin solution (D) | | | | | | | | D-1 | D-2 | D-3 |
| | | | | | | | | 0.5 | 0.5 | 0.5 |
| Chain transfer agent | | | | | | TPMB | PEMP | | | |
| | | | | | | 0.1 | 0.1 | | | |
| Leveling agent | 1.0 | 1.0 | 1.0 | 1.0 | 1.0 | 1.0 | 1.0 | 1.0 | 1.0 | 1.0 |
| Solvent 1 | S-1 | S-1 | S-1 | S-1 | S-1 | S-1 | S-1 | S-1 | S-1 | S-1 |
| | 34.2 | 34.2 | 34.2 | 34.2 | 34.2 | 34.5 | 34.5 | 34.5 | 34.5 | 34.1 |
| Solvent 2 | | | | | | | | | | |
| Total | 100.00 | 100.00 | 100.00 | 100.00 | 100.00 | 100.48 | 100.48 | 100.84 | 100.84 | 100.44 |
| NV (%) | 20.00 | 20.00 | 20.00 | 20.00 | 20.00 | 20.00 | 20.00 | 20.00 | 20.00 | 20.00 |

TABLE 2
Black photosensitive colored composition

| | Example | Example | Example | Example | Example | Example | Example | Example | Example | Example |
|---|---|---|---|---|---|---|---|---|---|---|
| | 8 | 9 | 10 | 11 | 12 | 13 | 14 | 15 | 16 | 17 |
| | R-11 | R-12 | R-13 | R-14 | R-15 | R-16 | R-17 | R-18 | R-19 | R-20 |
| Pigment dispersion (A) | PK-1 | PK-1 | PK-1 | PK-2 | PK-3 | PK-4 | PK-5 | PK-6 | PK-7 | PK-8 |
| | 56.0 | 56.0 | 56.0 | 56.0 | 56.0 | 56.0 | 56.0 | 56.0 | 56.0 | 56.0 |
| Photopolymerizable | B-1 | B-1 | B-1 | B-1 | B-1 | B-1 | B-1 | B-1 | B-1 | B-1 |
| monomer 1 | 3.0 | 3.0 | 3.0 | 3.0 | 3.0 | 3.0 | 3.0 | 3.0 | 3.0 | 3.0 |
| Photopolymerizable | B-2 | B-2 | B-2 | B-2 | B-2 | B-2 | B-2 | B-2 | B-2 | B-2 |
| monomer 2 | 3.0 | 3.0 | 3.0 | 3.0 | 3.0 | 3.0 | 3.0 | 3.0 | 3.0 | 3.0 |
| Photopolymerization | C-1 | C-1 | C-1 | C-1 | C-1 | C-1 | C-1 | C-1 | C-1 | C-1 |
| initiator (C) | 2.8 | 2.8 | 2.8 | 2.8 | 2.8 | 2.8 | 2.8 | 2.8 | 2.8 | 2.8 |
| Resin solution (D) | | | | | | | | | | |
| Chain transfer agent | | | | | | | | | | |
| Leveling agent | 1.0 | 1.0 | 1.0 | 1.0 | 1.0 | 1.0 | 1.0 | 1.0 | 1.0 | 1.0 |
| Solvent 1 | S-1 | S-1 | S-1 | S-1 | S-1 | S-1 | S-1 | S-1 | S-1 | S-1 |
| | 24.2 | 24.2 | 24.2 | 34.2 | 34.2 | 34.2 | 34.2 | 34.2 | 34.2 | 34.2 |
| Solvent 2 | S-2 | S-3 | S-4 | | | | | | | |
| | 10.0 | 10.0 | 10.0 | | | | | | | |
| Total | 100.00 | 100.00 | 100.00 | 100.00 | 100.00 | 100.00 | 100.00 | 100.00 | 100.00 | 100.00 |
| NV (%) | 20.00 | 20.00 | 20.00 | 20.00 | 20.00 | 20.00 | 20.00 | 20.00 | 20.00 | 20.00 |

TABLE 3
Black photosensitive colored composition

| | Example | Example | Example | Example | Example | Example | Example | Example | Example | Example |
|---|---|---|---|---|---|---|---|---|---|---|
| | 18 | 19 | 20 | 21 | 22 | 23 | 24 | 25 | 26 | 27 |
| | R-21 | R-22 | R-23 | R-24 | R-25 | R-26 | R-27 | R-28 | R-29 | R-30 |
| Pigment dispersion (A) | PK-9 | PK-10 | PK-11 | PK-12 | PK-13 | PK-1 | PK-1 | PK-1 | PK-1 | PK-1 |
| | 56.0 | 56.0 | 56.0 | 56.0 | 56.0 | 56.0 | 56.0 | 56.0 | 56.0 | 56.0 |
| Photopolymerizable | B-1 | B-1 | B-1 | B-1 | B-1 | B-1 | B-1 | B-1 | B-1 | B- |
| monomer 1 | 3.0 | 3.0 | 3.0 | 3.0 | 3.0 | 3.0 | 3.0 | 4.1 | 3.4 | 3.2 |
| Photopolymerizable | B-2 | B-2 | B-2 | B-2 | B-2 | B-2 | B-2 | B-2 | B-2 | B-2 |
| monomer 2 | 3.0 | 3.0 | 3.0 | 3.0 | 3.0 | 3.0 | 3.0 | 4.1 | 3.4 | 3.2 |
| Photopolymerization | C-1 | C-1 | C-1 | C-1 | C-1 | C-1 | C-1 | C-1 | C-1 | C-1 |
| initiator (C) | 2.8 | 2.8 | 2.8 | 2.8 | 2.8 | 2.8 | 2.8 | 2.8 | 2.8 | 2.8 |
| Resin solution (D) | | | | | | D-1 | D-1 | | | |
| | | | | | | 4.0 | 2.0 | | | |
| Chain transfer agent | | | | | | | | | | |
| Leveling agent | 1.0 | 1.0 | 1.0 | 1.0 | 1.0 | 1.0 | 1.0 | 1.0 | 1.0 | 1.0 |
| Solvent 1 | S-1 | S-1 | S-1 | S-1 | S-1 | S-1 | S-1 | S-1 | S-1 | S-1 |
| | 34.2 | 34.2 | 34.2 | 34.2 | 34.2 | 50.5 | 42.1 | 32.0 | 33.5 | 33.8 |
| Solvent 2 | | | | | | | | | | |
| Total | 100.00 | 100.00 | 100.00 | 100.00 | 100.00 | 120.27 | 109.90 | 100.00 | 100.07 | 100.00 |
| NV (%) | 20.00 | 20.00 | 20.00 | 20.00 | 20.00 | 20.00 | 20.00 | 20.00 | 20.00 | 20.00 |

TABLE 4

| | Example | Comparative | Comparative | Comparative |
|---|---|---|---|---|
| Black photosensitive | 28 | Example 4 | Example 5 | Example 6 |
| colored composition | R-31 | R-32 | R-33 | R-34 |
| Pigment dispersion (A) | PK-1 | PK-14 | PK-15 | PK-1 |
| | 56.0 | 56.0 | 56.0 | 56.0 |
| Photopolymerizable monomer | B-1 | B-1 | B-1 | B-1 |
| 1 | 2.8 | 3.0 | 3.0 | 3.0 |
| Photopolymerizable monomer | B-2 | B-2 | B-2 | B-2 |
| 2 | 2.8 | 3.0 | 3.0 | 3.0 |
| Photopolymerization initiator | C-1 | C-1 | C-1 | C-2 |
| (C) | 3.2 | 2.8 | 2.8 | 2.8 |
| Resin solution (D) | | | | |
| Chain transfer agent | | | | |
| Leveling agent | 1.0 | 1.0 | 1.0 | 1.0 |
| Solvent 1 | S-1 | S-1 | S-1 | S-1 |
| | 34.2 | 34.2 | 34.2 | 34.2 |
| Solvent 2 | | | | |
| Total | 100.00 | 100.00 | 100.00 | 100.00 |
| NV (%) | 20.00 | 20.00 | 20.00 | 20.00 |

The abbreviations in the tables are shown below.

### <Polymerizable Compound>

Polymerizable compound B-1: Dipentaerythritol hexaacrylate (“ARONIX M-402” manufactured by Toagosei Co., Ltd.)
Polymerizable compound B-2: Trimethylolpropane EO-modified triacrylate (“ARONIX M-350” manufactured by Toagosei Co., Ltd.)
Polymerizable compound B-3: Trimethylolpropane triacrylate (“ARONIX M-309” manufactured by Toagosei Co., Ltd.)

### <Photopolymerization Initiator>

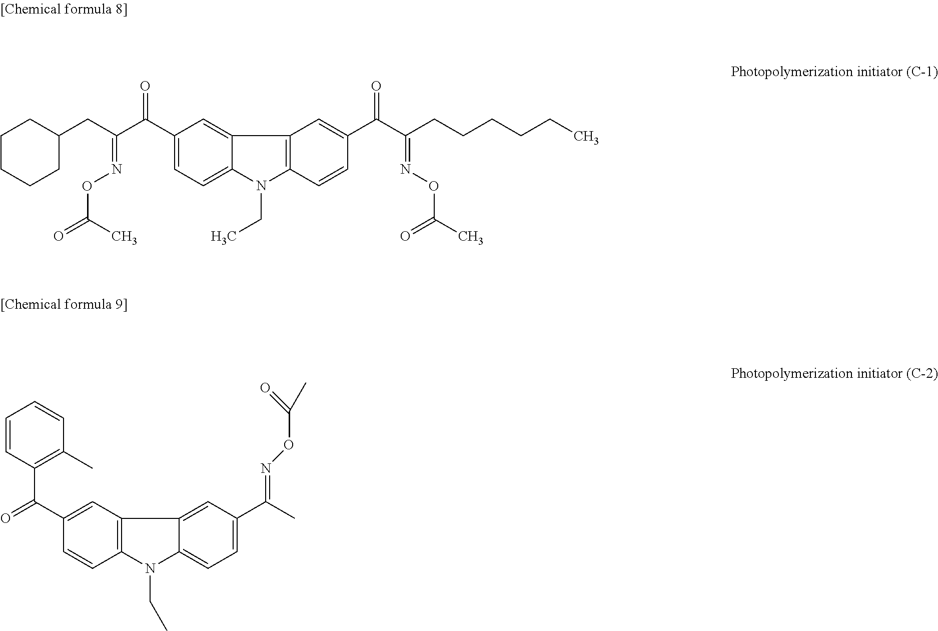 [SMILES: CCCCCC/C(=N\OC(C)=O)C(=O)c1ccc2c(c1)c1cc(C(=O)/C(CC3CCCCC3)=N/OC(C)=O)ccc1n2CC ; CCn1c2ccc(C(=O)c3ccccc3C)cc2c2cc(/C(C)=N/OC(C)=O)ccc21]

### <Leveling Agent>

Leveling agent: A solution prepared by dissolving 1 part of “FZ-2122” manufactured by Toray-Dow Corning in 99 parts of propylene glycol monomethyl ether acetate (PGMAc)

### <Chain Transfer Agent>

TPMB: Trimethylolpropane tris(3-mercaptobutyrate) (“TPMB” manufactured by Showa Denko K.K.)
PEMP: Pentaerythritol tetrakis(3-mercaptopropionate) (manufactured by SC Organic Chemical Co., Ltd.)

### <Solvent>

Solvent S-1: Propylene glycol monomethyl ether acetate
Solvent S-2: Cyclohexanone
Solvent S-3: Tripropylene glycol monomethyl ether
Solvent S-4: 3-methoxy-1-butanol

### <Optical Density>

The black photosensitive composition was applied to a 100 mm×100 mm glass substrate by spin coating, and then a coating film with a dry film thickness of 1.2 μm was prepared and heated at 100° C. for 5 minutes. The optical density (OD) of the black photosensitive composition-coated substrate obtained in this manner was measured using a Macbeth densitometer (GRETAGD200-II), and the optical density (OD) at a film thickness of 1.2 μm was determined. In addition, the OD was evaluated on a 3-point scale according to the following criteria.
A: 2.7 or more
B: 2.5 or more and less than 2.7
C: Less than 2.5

### <Pixel Shape (Linearity)>

Each photosensitive composition used in the examples was applied to a 200 mm diameter silicon wafer substrate using a spin coater to form a thickness of 1.00 μm after drying, and dried at 70° C. for 1 minute to obtain a substrate. Next, exposure was performed at 8000 J/m² with a wavelength of 365 nm using an i-line stepper exposure device FPA-3000i4 (manufactured by Canon). The exposure was performed through a photomask having square openings of 400 μm on each side. The film after exposure was paddle developed for 1 minute with TMAH 2.38% (2.38% aqueous solution of tetramethylammonium hydroxide manufactured by Tama Chemicals). After paddle development, rinsing was performed with pure water by spin shower for 20 seconds, followed by spin drying. The linearity of the pixels formed by the openings was observed using a scanning electron microscope (S-3000N manufactured by Hitachi High-Tech) and evaluated according to the following criteria.
A: Pixel linearity is good throughout the entire surface (preferable level for practical use)
B: Pixel linearity is partially good (usable level in practice)
C: Pixel linearity is poor throughout the entire surface (level unsuitable for practical use)

### <Pixel Shape (Cross-Section)>

Each photosensitive composition used in the examples was applied to a 200 mm diameter silicon wafer substrate using a spin coater to form a thickness of 1.00 μm after drying, and dried at 70° C. for 1 minute to obtain a substrate. Next, exposure was performed at 8000 J/m² with a wavelength of 365 nm using an i-line stepper exposure device FPA-3000i4 (manufactured by Canon). The exposure was performed through a photomask having square openings of 5 mm on each side. The film after exposure was paddle developed for 1 minute with TMAH 2.38% (2.38% aqueous solution of tetramethylammonium hydroxide manufactured by Tama Chemicals). After paddle development, rinsing was performed with pure water by spin shower for 20 seconds, followed by spin drying. The pixels formed by the openings were cut out, and the cross-section was observed using a scanning electron microscope (S-3000N manufactured by Hitachi High-Tech) and evaluated according to the following criteria.
A: The pixel edge is in close contact with the substrate (preferable level for practical use)
B: The lifting of the pixel edge is less than 3 μm (usable level in practice)
C: The lifting of the pixel edge is 3 μm or more (level unsuitable for practical use)

### <Developability (Residue)>

Each photosensitive composition was applied to a 200 mm diameter silicon wafer substrate using a spin coater to form a thickness of 1.2 μm after drying, and dried at 70° C. for 1 minute to obtain a substrate. Next, exposure was performed at 8000 J/m² with a wavelength of 365 nm using an i-line stepper exposure device FPA-3000i4 (manufactured by Canon). The exposure was performed through a photomask having square openings of 400 μm on each side. The film after exposure was paddle developed for 1 minute with TMAH 2.38% (2.38% aqueous solution of tetramethylammonium hydroxide manufactured by Tama Chemicals). After paddle development, rinsing was performed with pure water by spin shower for 20 seconds, followed by spin drying. The development residue in the unexposed portions was observed using a scanning electron microscope (S-3000N manufactured by Hitachi High-Tech) and evaluated according to the following criteria.
A: No development residue in the unexposed portions (preferable level for practical use)
B: Development residue exists in some parts of the unexposed portions (usable level in practice)
C: Development residue exists over the entire unexposed portions (level unsuitable for practical use)

### <Solvent Resistance>

Each photosensitive composition was applied to a 200 mm diameter silicon wafer substrate using a spin coater to form a thickness of 1.2 μm after drying, and dried at 70° C. for 1 minute to obtain a substrate. Next, exposure was performed at 8000 J/m² with a wavelength of 365 nm using an i-line stepper exposure device FPA-3000i4 (manufactured by Canon). The exposure was performed through a photomask having square openings of 400 μm on each side. The film after exposure was paddle developed for 1 minute with TMAH 2.38% (2.38% aqueous solution of tetramethylammonium hydroxide manufactured by Tama Chemicals). After paddle development, rinsing was performed with pure water by spin shower for 20 seconds, followed by spin drying. After spin drying, the substrate was baked on a hot plate at 100° C. for 5 minutes to obtain a black pattern substrate. The film thickness of the obtained pattern was measured using a contact-type film thickness meter (Dektak150 manufactured by Bruker Japan). After measuring the film thickness, the substrate was immersed in methoxy propyl acetate at 23° C. for 5 minutes, and then the film thickness was measured again and evaluated according to the following criteria.
A: Change in film thickness before and after immersion is less than 5%
B: Change in film thickness before and after immersion is 5% or more and less than 10%
C: Change in film thickness before and after immersion is 10% or more

### <Storage Stability>

The initial viscosity was measured the day after preparing the curable resin composition, and the aged viscosity was measured after accelerated aging at 40° C. for 1 week. From the obtained initial viscosity and aged viscosity values, the aged viscosity change rate was calculated according to the formula below, and the storage stability was evaluated according to the following criteria.

[Aged viscosity change rate]=|([Initial viscosity]−[Aged viscosity])/[Initial viscosity]|×100

The measurement conditions were as follows: with use of an E-type viscometer (TV-25 type viscometer manufactured by Toki Sangyo Co., Ltd.), measurements were performed at 25° C., with rotor No. 1, measurement time of 3 minutes, and rotation speed of 20 rpm.
A: Change rate less than 5%
B: Change rate 5% or more and less than 10%
C: Change rate 10% or more

### <Development Adhesion>

Each photosensitive composition was applied to a 200 mm diameter silicon wafer substrate using a spin coater to form a thickness of 1.2 μm after drying, and then dried at 70° C. for 1 minute to obtain a substrate. Next, exposure was performed at 8000 J/m² with a wavelength of 365 nm using an i-line stepper exposure device FPA-3000i4 (manufactured by Canon). The exposure was performed 10 times at different positions on the silicon wafer substrate through a photomask having square openings of 400 μm on each side. The film after exposure was paddle developed for 1 minute with TMAH 2.38% (2.38% aqueous solution of tetramethylammonium hydroxide manufactured by Tama Chemicals). After paddle development, rinsing was performed with pure water by spin shower for 20 seconds, followed by spin drying, and the pixel retention rate of the exposed portions was observed using a scanning electron microscope (S-3000N manufactured by Hitachi High-Tech) and evaluated according to the following criteria.
A: Number of remaining pixels is 10 (preferable level for practical use)
B: Number of remaining pixels is 8 to 9 (usable level in practice)
C: Number of remaining pixels is less than 8 (level unsuitable for practical use)

The results of the above evaluations are shown in Table 5 to Table 8.

TABLE 5

| | Comparative | Comparative | Comparative | Example | Example | Example | Example | Example | Example | Example |
|---|---|---|---|---|---|---|---|---|---|---|
| | Example 1 | Example 2 | Example 3 | 1 | 2 | 3 | 4 | 5 | 6 | 7 |
| | R-1 | R-2 | R-3 | R-4 | R-5 | R-6 | R-7 | R-8 | R-9 | R-10 |
| Film thickness [μm] | 1.2 | 1.2 | 1.2 | 1.2 | 1.2 | 1.2 | 1.2 | 1.2 | 1.2 | 1.2 |
| Carbon black | 24 | 24 | 24 | 24 | 24 | 24 | 24 | 24 | 24 | 24 |
| primary particle | | | | | | | | | | |
| diameter [nm] | | | | | | | | | | |
| Carbon black | 115 | 115 | 115 | 115 | 115 | 115 | 115 | 115 | 115 | 115 |
| specific surface | | | | | | | | | | |
| area [m²/g] | | | | | | | | | | |
| OD | A | A | A | A | A | A | A | A | A | A |
| Pixel shape | B | B | B | A | A | A | A | B | B | B |
| (linearity) | | | | | | | | | | |
| Pixel shape | B | B | B | B | B | A | A | B | B | B |
| (cross-section) | | | | | | | | | | |
| Developability | B | A | B | A | B | B | B | A | A | A |
| (residue) | | | | | | | | | | |
| Solvent resistance | A | B | A | A | A | A | A | A | B | B |
| Storage stability | A | A | A | A | A | A | A | A | A | A |
| Development | A | A | A | A | A | A | A | A | A | B |
| adhesion | | | | | | | | | | |

TABLE 6

| | Example | Example | Example | Example | Example | Example | Example | Example | Example | Example |
|---|---|---|---|---|---|---|---|---|---|---|
| | 8 | 9 | 10 | 11 | 12 | 13 | 14 | 15 | 16 | 17 |
| | R-11 | R-12 | R-13 | R-14 | R-15 | R-16 | R-17 | R-18 | R-19 | R-20 |
| Film thickness [μm] | 1.2 | 1.2 | 1.2 | 1.2 | 1.2 | 1.2 | 1.2 | 1.2 | 1.2 | 1.2 |
| Carbon black primary | 24 | 24 | 24 | 24 | 24 | 25 | 23 | 27 | 24 | 30 |
| particle diameter [nm] | | | | | | | | | | |
| Carbon black specific | 115 | 115 | 115 | 120 | 125 | 120 | 132 | 88 | 110 | 74 |
| surface area [m²/g] | | | | | | | | | | |
| Optical density (OD) | A | A | A | A | A | A | A | B | A | B |
| Pixel shape (linearity) | A | A | A | A | A | A | A | A | A | A |
| Pixel shape (cross-section) | B | A | A | B | B | B | B | A | B | B |
| Developability (residue) | A | A | A | A | A | A | B | A | A | A |
| Solvent resistance | B | B | B | A | A | A | A | A | A | A |
| Storage stability | A | A | A | A | A | A | A | A | A | A |
| Development adhesion | A | A | A | A | A | A | A | A | A | A |

TABLE 7

| | Example | Example | Example | Example | Example | Example | Example | Example | Example | Example |
|---|---|---|---|---|---|---|---|---|---|---|
| | 18 | 19 | 20 | 21 | 22 | 23 | 24 | 25 | 26 | 27 |
| | R-21 | R-22 | R-23 | R-24 | R-25 | R-26 | R-27 | R-28 | R-29 | R-30 |
| Film thickness [μm] | 1.2 | 1.2 | 1.2 | 1.2 | 1.2 | 1.2 | 1.2 | 1.2 | 1.2 | 1.2 |
| Carbon black primary | 76 | 18 | 24 | 24 | 24 | 24 | 24 | 24 | 24 | 24 |
| particle diameter [nm] | | | | | | | | | | |
| Carbon black specific | 29 | 180 | 115 | 115 | 115 | 115 | 115 | 115 | 115 | 115 |
| surface area [m²/g] | | | | | | | | | | |
| OD | B | A | A | A | A | A | A | A | A | A |
| Pixel shape (linearity) | A | A | B | B | B | B | B | B | A | A |
| Pixel shape (cross-section) | A | A | B | B | B | B | B | B | B | A |
| Developability (residue) | A | A | A | A | A | A | A | B | B | B |
| Solvent resistance | A | A | A | B | B | B | A | A | A | A |
| Storage stability | A | B | A | A | A | A | A | A | A | A |
| Development adhesion | B | A | A | A | B | A | A | A | A | A |

TABLE 8

| | Example | Comparative | Comparative | Comparative |
|---|---|---|---|---|
| | 28 | Example 4 | Example 5 | Example 6 |
| | R-31 | R-33 | R-34 | R-6 |
| Film thickness [μm] | 1.2 | 1.2 | 1.2 | 1.2 |
| Carbon black primary particle | 24 | — | — | 24 |
| diameter [nm] | | | | |
| Carbon black specific surface | 115 | — | — | 115 |
| area [m²/g] | | | | |
| OD | A | C | C | A |
| Pixel shape (linearity) | A | A | A | A |
| Pixel shape (cross-section) | A | A | A | C |
| Developability (residue) | A | A | A | A |
| Solvent resistance | A | A | A | C |
| Storage stability | B | A | A | A |
| Development adhesion | A | A | A | A |

The photosensitive compositions for organic EL display device of Examples 1 to 28, which include carbon black, a polymerizable compound, a photopolymerization initiator including a compound having two or more oxime esters in one molecule, and a photosensitive resin, wherein the polymerizable compound includes a hexafunctional polymerizable compound and a trifunctional polymerizable compound, and the carbon black is 30% by mass to 50% by mass in non-volatile content, were shown to have excellent preservation stability, excellent curability at a low temperature, good developability, and the ability to form a cured film having high light-shielding properties.

### INDUSTRIAL APPLICABILITY

The present invention can be used as a black photosensitive composition for organic EL display device, for electronic devices such as smart glasses, head-mounted displays, and electronic viewfinders.

This application claims priority based on Japanese Patent Application No. 2022-101262, filed on Jun. 23, 2022, the entire disclosure of which is incorporated herein by reference.

### REFERENCE SIGNS LIST

10: substrate, 20: pixel electrode, 30: organic EL layer, 40: color filter layer, 100: organic EL display device, 4R: red pixel, 4G: green pixel, 4B: blue pixel, 3: partition wall, 5: black matrix (light-shielding layer), 32: sealing layer, 34: adhesive layer, 42: overcoat layer, 44: adhesive layer, 46: protective layer

1. A photosensitive composition for organic EL display device, comprising carbon black, a polymerizable compound, a photopolymerization initiator, and a photosensitive resin,
wherein the photopolymerization initiator comprises a compound having two or more oxime esters in one molecule,
the polymerizable compound comprises a hexafunctional polymerizable compound and a trifunctional polymerizable compound, and
the photosensitive composition for organic EL display device comprises 30% by mass to 50% by mass of the carbon black in a non-volatile content of the photosensitive composition for organic EL display device.

2. The photosensitive composition for organic EL display device according to claim 1, wherein the compound having two or more oxime esters in one molecule comprises a compound represented by the following chemical formula (1-1).

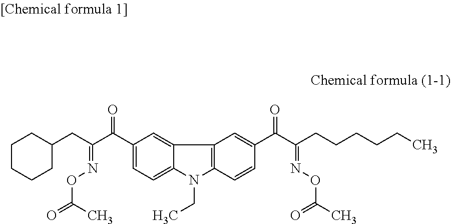 [SMILES: CCCCCC/C(=N\OC(C)=O)C(=O)c1ccc2c(c1)c1cc(C(=O)/C(CC3CCCCC3)=N/OC(C)=O)ccc1n2CC]

3. The photosensitive composition for organic EL display device according to claim 1, wherein an average primary particle diameter of the carbon black is 50 nm or less.

4. The photosensitive composition for organic EL display device according to claim 1, wherein a specific surface area of the carbon black is 80 m²/g to 260 m²/g.

5. The photosensitive composition for organic EL display device according to claim 1, comprising 3% by mass to 16% by mass of the photopolymerization initiator in the non-volatile content of the photosensitive composition for organic EL display device.

6. The photosensitive composition for organic EL display device according to claim 1, wherein a ratio of the photosensitive resin is 80% by mass or more in all resin components contained in the photosensitive composition.

7. An organic EL display device, comprising a light-shielding layer formed from the photosensitive composition for organic EL display device according to claim 1.
